SEQUENCE LISTING (9 sequences)

SEQ ID NO: 1 - protein, 12 aa, Artificial Sequence
  Synthetic Peptide
QXEAGXLXKXKX

SEQ ID NO: 2 - protein, 15 aa, Artificial Sequence
  Synthetic antimicrobial peptide (Cecropin- Melittin Hybrid)
KWKLFKKIGAVLKVL

SEQ ID NO: 3 - protein, 20 aa, Artificial Sequence
  Synthetic antimicrobial sequence (D28)
FLGVVFKLASKVFPAVFGKV

SEQ ID NO: 4 - protein, 20 aa, Artificial Sequence
  Synthetic antimicrobial peptide (D51)
FLFRVASKVFPALIGKFKKK

SEQ ID NO: 5 - protein, 22 aa, Artificial Sequence
  Synthetic antimicrobial peptide (MICL-1)
GIGKFLKKAKKFGKAFVKILKK

SEQ ID NO: 6 - protein, 29 aa, Artificial Sequence
  Synthetic antimicrobial sequence (MICL-41)
RGLRRLGRKIAHGVKKYGPTVLRIIRIAG

SEQ ID NO: 7 - protein, 30 aa, Artificial Sequence
  Synthetic antimicrobial peptide (MICL-42)
GWKDWAKKAGGWLKKKGPGMAKAALKAAMQ

SEQ ID NO: 8 - protein, 16 aa, Artificial Sequence
  Synthetic cysteine-incorporating Cecropi- Melittin hybrid peptide
KWKLFKKIGAVLKVLC

SEQ ID NO: 9 - protein, 16 aa, Artificial Sequence
  Synthetic cysteine-incorporating Cecropin- melittin hybrid peptide
KWKLFKKIGAVLKVLC

(19) United States
(12) Patent Application Publication
(10) Pub. No.: US 2009/0155335 A1
(43) Pub. Date: Jun. 18, 2009

(54) NON-LEACHING NON-FOULING ANTIMICROBIAL COATINGS

(75) Inventors: William Shannan O'Shaughnessey, Boston, MA (US); Zheng Zhang, Cambridge, MA (US); Michael Hencke, Cambridge, MA (US); Kris Wood, Cambridge, MA (US); Trevor Squier, Peabody, MA (US); Christopher R. Loose, Cambridge, MA (US)

Correspondence Address: Pabst Patent Group LLP, 1545 PEACHTREE STREET NE, SUITE 320, ATLANTA, GA, 30309, US

(73) Assignee: Semprus Biosciences Corp.

(21) Appl. No.: 12/329,344

(22) Filed: Dec. 5, 2008

Related U.S. Application Data
(60) Provisional application No. 60/992,629, filed on Dec. 5, 2007.

(51) Int. Cl. A61L 29/16; A61K 38/16; A61K 38/10; C07K 14/00; C07K 7/04; A61L 15/44; A61L 31/16; A61L 27/54; A61P 31/04

(52) U.S. Cl. 424/423; 514/13; 514/12; 514/14; 530/324; 530/326; 530/327

(57) ABSTRACT

Compositions containing one or more types of membrane-targeting antimicrobial agents immobilized on a substrate with activity in relevant biological environments, and methods of making and using thereof, are described herein. The antimicrobial agents retain their activity in the presence of blood proteins and/or in vivo due to improved molecular structures which allow for cooperative action of immobilized agents and hydrophilic chemistries which resist non-specific protein adsorption. Suitable molecular structures include branched structures, such as dendrimers and randomly branched polymers. The molecule structures may also include hydrophilic tethers which provide both flexibility and resistance to non-specific protein adsorption. The membrane targeting antimicrobial agent coatings can be applied to a variety of different types of substrates including medical implants such as vascular grafts, orthopedic devices, dialysis access grafts, and catheters; surgical tools, surgical garments; and bandages. The substrates can be composed of metallic materials, ceramics, polymers, fibers, inert materials such as silicon, and combinations thereof. The compositions described herein are substantially non-leaching, resistant to non-specific protein adsorption, and non-hemolytic.

### CROSS-REFERENCE TO RELATED APPLICATIONS

This application claims benefit of U.S. Ser. No. 60/992,629, entitled “Immobilized Antimicrobial Coatings” by William Shannan O'Shaughnessey, Christopher R. Loose, Michael Hencke, Kris Wood, Trevor Squier, and Zheng Zhang, filed in the U.S. Patent and Trademark Office on Dec. 5, 2007 which is incorporated by referenced in its entirety.

### GOVERNMENT SUPPORT

This invention was made with Government support under Grant No. SBIR #0712010 awarded by the National Science Foundation to Semprus BioSciences. The Government has certain rights in this invention.

### FIELD OF THE INVENTION

The present invention is generally in the field of immobilized antimicrobial coatings, specifically coatings which exhibit bacteristatic and bactericidal properties without leaching of the active agent. The efficacy of the coatings is optimized by use of molecular architectures which, through both their structure and chemical composition, maximize antimicrobial functionality in the presence of biological fluids and within the in vivo environment.

### BACKGROUND OF THE INVENTION

Nosocomial infections are becoming increasingly costly and difficult to treat due to the spread of drug resistant bacteria. Despite efforts to improve the sterility of surgical procedures, infection remains common. These infections are often associated with medical devices. Devices which penetrate the skin and/or are inserted into the body via a body cavity or orifice, such as central venous catheters and urinary catheters, can provide a route for bacteria to enter the body, and implanted devices form favorable surfaces on which bacteria can grow.

Once bacteria colonize a medical device, they may form a recalcitrant biofilm. A biofilm is a complex aggregation of microorganisms marked by the excretion of a protective and adhesive matrix. Biofilms are also often characterized by surface attachment, structural heterogeneity, genetic diversity, complex community interactions, and an extracellular matrix of polymeric substances. The biofilm protects bacteria in the interior of the film from the immune system. Systemic antibiotics are ineffective in treating such infections due to their limited ability to penetrate biofilms. For these reasons, the treatment of device infections often involves the removal of the device and administration of antibiotics, followed by the insertion of a new device. This procedure may be costly and painful, and if the bacteria are not completely cleared, the new device may become infected.

A variety of controlled-release antimicrobial coatings and devices have been developed, particularly for devices such as central venous catheters (CVCs) and wound dressings, for which bacterial infection is especially problematic. Existing antimicrobial coatings generally consist of antibiotic agents or metal ions absorbed onto the device surface, or incorporated into a polymer coating or into the bulk device material. Slow release of these agents results in localized toxic concentrations that help reduce bacterial colonization and proliferation.

There are currently three commercially available antimicrobial CVCs. ARROWg+ard® Blue catheters (Arrow International) are impregnated with a combination of chlorhexidine (Kuyyakanond, et at, FEMS Micro. Let., 100(1-3), 211-215 (1992)) and silver sulfadiazine, whose antimicrobial activity is primarily due to silver's disruption of the electron transport chain and DNA replication (Silver et al., J. Ind. Micro. Biotech., 33(7), 627-634 (2006); Fox et al., Antimicrob. Agents & Chemotherapy, 5(6), 582-588 (1974)). These catheters have been shown in clinical studies to reduce catheter colonization by 44% (Veenstra et al., J. Amer. Med. Assoc., 281(3), 261-267 (1999)). Chlorhexidine, however, is known to result in hypersensitivity reactions in patients (Wu et al., Biomaterials, 27(11):2450-67 (2006)), and both chlorhexidine and silver sulfadiazine may induce bacterial resistance (Brooks et al., Inf. Con. Hos. Epidem., 23(11): 692-695 (2002); Silver et al., J. Ind. Micro. Biotech., 33(7), 627-634 (2006)).

Cook Critical Care's Spectrum® line of catheters utilizes the slow release of minocycline, which disrupts protein synthesis (Speers et al., Clin. Microbio. Rev., 5(4): 387-399 (1992)) and rifampin, which inhibits RNA polymerase (Kim et al., Sys. Appl. Microbiol., 28(5): 398-404 (2005)). These catheters have been shown in clinical studies to reduce catheter colonization by 69% (Raad et al., Ann. Int. Med., 127(4): 267 (1997)). However, minocycline and rifampin are also known to induce bacterial resistance (Kim et al, Sys. Appl. Microbiol., 28(5): 398-404 (2005); Speers et al, Clin. Microbio. Rev., 5(4): 387-399 (1992)).

Edwards Lifesciences' Vantex® catheters release silver, carbon, and platinum ions, with most of the antimicrobial activity attributed to the silver ions. These catheters have a demonstrated reduction in catheter colonization of approximately 35%, which may be limited in part by the in vivo sequestration of silver ions by albumin in the blood stream (Ranucci et al., Crit. Care Med., 31(1): 52-59 (2003); Corral et al., J. Hos. Infec., 55(3): 212-219 (2003)). Bacterial resistance to silver ions has also been reported (Silver et al., J. Ind. Micro. Biotech., 33(7), 627-634 (2006)). A number of antimicrobial wound dressings have also been developed, with the majority based on the incorporation of silver ions, such as ConvaTec's Aquacel®. Other antimicrobial agents include cadexomer iodine (Smith & Nephew's Iodoflex™ and Iodosorb™), chlorhexidine gluconate (CHG, (Johnson & Johnson's Biopatch™), and polyhexamethylene biguanide (PHMB, Kendall Healthcare's Kerlix™ AMD™).

An attractive alternative to these agents are antimicrobial peptides (AmPs). AmPs can distinguish between mammalian cells and microbes based on membrane properties, and kill microbes using a fast and non-specific mechanism of attack. While the mechanism of AmP-induced membrane destabilization has yet to be fully described, current theories all involve establishment of a threshold concentration of AmP on the surface of the bacterial membrane. This is followed by cooperative action of the AmP molecules to either permeate or otherwise destroy the membrane (Y. Shai, Biopolymers (peptide science) Vol. 66, 236-248 (2002)). This mechanism is thought to be dramatically less likely to induce drug resistance as compared to antibiotics that target specific enzymes because the evolutionary cost for changing membrane properties is greater and the attack is sufficiently fast that bacteria have little opportunity to survive and mutate.

Naturally occurring AmPs may have activity against Gram positive and negative bacteria, fungi, and viruses. It has been shown that releasing AmPs from the surface of a device has the ability to prevent device related infections. Soaking a Dacron® graft in a solution of the AmP dermaseptin before implanting it in a rat and challenging with bacteria, reduces the incidence of device colonization and infection (Balaban et al., Antimicrob. Agents & Chemother., 48: 2544-2550 (2004)). The release of dermaseptin was effective against both methicillin resistant and vancomycin intermediate-resistant Staphylococcus aureus. Migenix and Cadence's antimicrobial peptide drug candidate CPI-226 has shown in vivo efficacy in a slow release cream formulation in clinical trials against bacteria associated with medical device infection.

All slow-release coatings (including those using small molecule antimicrobials, metal ions, AMPs, and other agents described above) suffer from several inherent limitations. By design, they have a limited lifespan, and for many catheter applications, including CVCs and dialysis catheters, extended protection is desired by clinicians. Additionally, slowly released antibiotics create neighboring regions of sub-lethal drug concentrations that may encourage the development of drug resistance. By releasing drugs into the bloodstream, there are also increased concerns regarding systemic toxicity. The toxicity concerns also lead to an increased clinical safety and regulatory burden in developing these technologies. Further, due to the large loading of drug that may be required to create a slow release coating, the structural and performance properties of the device may be impacted.

In contrast, non-leaching antimicrobial surfaces have the potential to provide long-lasting protection from bacterial colonization and biofilm formation without the side effects caused by systemic distribution of antimicrobial agents. Such surfaces have been created using quaternary ammonium compounds, which generally combine cationic groups with hydrophobic chains. These chains work to disrupt bacterial membranes (Lewis et al., Trends in Biotech., 23(7): 343-348 (2005)). Surfaces coated with high molecular weight polymers presenting these groups have been demonstrated to have immobilized antimicrobial efficacy. However, these compounds have insufficient therapeutic indices for use on implanted medical devices, as they typically exhibit high hemolytic activities. Additional amphipathic membrane-targeting antimicrobial agents that mimic the action of AmPs have also been developed, most notably by Tew and coworkers (Gabriel et al., Mat. Sci. & Eng. R, 57: 28-64 (2007)). These tend to be smaller oligomers, generally less than 2000 daltons, that are both amphiphilic and cationic, similar to AmPs made up of natural amino acids. While these materials demonstrate higher therapeutic indices than those observed with high molecular weight quaternary ammonium polymers, they have yet to be shown to be effective in immobilized coatings.

Non-leaching antimicrobial surfaces must not only possess high levels of surface antimicrobial activity, but must also avoid biological fouling which can lead to complications including loss of antimicrobial activity (by blocking physical access of the surface to microbes) and thrombosis. It is known that medical devices that are introduced into environments where they contact complex biological fluids, such as blood, can non-specifically adsorb proteins from these fluids onto their surfaces. These proteins adhere to the device surface and denature, generally due to nonspecific hydrophobic interactions (Andrade et al., Adv. in Polymer Sci., 79: 1-63 (1986)). These adsorption processes have been shown to contribute to thrombosis in bloodstream environments (Bailly et al., J. Biomed. Mat. Res., 30(1): 101-108 (1996)) as well as adhesion of various bacterial species (Harris et. al., Int. J. Care Injured, 37: S3-S14 (2006)). Attempts to create immobilized antimicrobial peptide coatings have been reported in the literature (see U.S. Pat. No. 5,847,047 to Haynie; U.S. Patent Application Publication No. 2005/0065072 by Keeler et al.; U.S. Patent Application Publication No. 2004/0126409 by Wilcox et al.; and European Patent No. EP 0 990 924 to Johnson and Johnson). However, the issues of fouling and/or thrombosis formation have not been addressed. As a result, the formulations may adsorb biological proteins in vivo, which may block the availability of immobilized peptides to interact with bacteria and potentially decreasing the efficacy of these formulations. In addition to peptide based coatings, hydrophobic quaternary ammonium compounds are particularly susceptible to protein fouling in a blood environment, reducing their antimicrobial efficacy. WO07084452 by Hydromer attempts to overcome this limitation by making quaternary ammonium chains of sufficient length to penetrate through any deposited protein layer or cell debris. Regardless of length, however, these hydrophobic chains adsorb proteins and cell debris rather than cleanly passing through the debris to bacteria above.

The chemical nature of the materials encountering a protein solution has a significant influence on the nonspecific adsorption of the proteins. Multiple materials have been developed with the goal of resisting non-specific protein adsorption including: poly(ethylene glycol) (PEG) based materials, dextran and other sugar based materials, and zwitterionic materials (Ratner et al., Annual Review of Biomed. Eng., 6: 41-75 (2004); Österberg et al. J. Biomed. Mat. Res., 29: 741-747 (1995); Zhang et al., Langmuir, 22(24): 1072-1077 (2006)). Some of these, such as dextran-based materials, induce a negative response as they may not resist non-specific protein adsorption to a large enough degree. The use of appropriate molecular architectures or tethering may also enhance the activity of tethered membrane targeting antimicrobials. Zwitterionic phosphorylcholine (PC) is a component of the outside layer of cell membranes. PC-based polymers such as poly(2-methacryloyloxyethyl phosphorylcholine) (polyMPC) can decrease nonspecific protein adsorption (Ishihara et al., J. Biomed. Mater. Res., 25: 1397-1407 (1991). Zwitterionic polymers such as sulfobetaine- and carboxybetaine-based polymers are highly resistant to protein adsorption, especially the protein adsorption in a complex media such as plasma and serum (Zhang et al., J. Phys. Chem. B 110: 10799-10804 (2006) and Biomaterials 29: 4285-4291 (2008)). These zwitterionic polymers may exhibit better stability than PC-based polymers. The latter may be degradable through hydrolysis or by phospholipase (Wang et al. Biomaterials 24, 3969-3980 (2003)).

In addition to enhancing the activity of immobilized antimicrobial agents by preventing nonspecific adsorption of proteins, zwitterionic moieties can also prevent attachment of bacteria to the surface on which they are attached. The bacteriostatic properties of the zwitterionic moiety can work in tandem with immobilized antimicrobial agents to create surfaces highly resistant to bacterial biofilm formation. Additionally, zwitterionic surfaces are thrombus resistant, and it is believed that thrombus may play a role in colonization and infection. By reducing thrombus formation, zwitterionic surfaces can work with AmPs to inhibit or prevent colonization through an additional mechanism of action. It would be desirable to have a single surface modification that prevents both infection and thrombosis for many devices that suffer from both of these complications including, but not limited to, vascular access, stents, grafts, valves, and other devices contacting the bloodstream.

There exists a need for antimicrobial compositions, particularly antimicrobial surfaces, with enhanced efficacy in preventing microbial attachment and proliferation. Specifically, there is a need for antimicrobial medical devices which retain their activity and are stable in the presence of fouling environments, such as in the presence of blood proteins, and/or in vivo.

It is therefore an object of the invention to provide a material having enhanced efficacy in preventing microbial attachment and proliferation.

It is further an object of the invention to provide antimicrobial formulations, which retain their activity in the presence of blood proteins and/or in vivo so they remain active and stable in vivo.

### SUMMARY OF THE INVENTION

Compositions containing one or more membrane-targeting antimicrobial agents immobilized on a substrate, in combination with a non-fouling material which can be coated onto or form all or part of the substrate, and methods of making and using thereof, have been developed. These provide greater efficacy in environments in which the compositions are exposed to cells, tissues or bodily fluids by providing enhanced antimicrobial and anti-fouling properties. The membrane-targeting antimicrobial agents, preferably antimicrobial peptides, target the membranes of the bacteria. Unlike most traditional antibiotics, which must be released to reach their targets in the interior of bacterial cells, membrane targeting antimicrobials must only contact the outer membrane or cell wall of the bacteria to be effective. The antimicrobial agents are covalently incorporated into molecular architectures on the substrate directly, via tethers to the substrate, and/or via the anti-fouling material (which may function as a tether). The immobilized antimicrobial agents retain sufficient flexibility and mobility to interact with the bacteria, viruses, and/or fungi upon surface exposure. The efficacy of the immobilized antimicrobial agents is maximized by varying the structure and chemical composition of the molecular architecture (i.e., the reactive groups on the substrate, the tethers coupling the antimicrobial agents to the substrate, and/or the anti-fouling material). Structures include polymers with varying tacticity and configuration, including, but not limited to, atactic, isotactic syndiotactic, diads, triads, tetrads, pentads, higher order tacticity configurations and any combinations thereof, branched structures, such as dendrimers and randomly branched polymers, and polymer brushes useful for presenting immobilized antimicrobials in a manner allowing multivalent interactions with bacteria. Additional structures include protein resistant tethers which provide both flexibility and resistance to non-specific protein adsorption. Zwitterionic surfaces have demonstrated an ability to modulate part of the foreign body reaction to biomaterials by means of their ability to resist non-specific protein adsorption and attachment. The ability to reduce non-specific protein adsorption/attachment and thrombus allows the zwitterionic surfaces to reduce the potential of fouling and blockage of the membrane targeting antimicrobials. Other chemical structures which combine tethered antimicrobials and non-specific protein adsorption resistant materials for increased antimicrobial efficacy and in vivo durability are also disclosed.

The membrane targeting antimicrobial agent coatings can be applied to a variety of different types of substrates including, but not limited to, surgical, medical or dental devices, instruments and/or implants. The substrates can be composed of metallic materials, ceramics, polymers, woven and non-woven materials, such as natural and synthetic fibers, inert materials such as silicon, and combinations thereof. The compositions are substantially non-leaching of immobilized membrane targeting antimicrobial agent, resistant to non-specific protein adsorption, and non-hemolytic. In addition, the antimicrobial agents remain active and stable both in vivo and when exposed in vitro to complex biological fluids, such as blood. Immobilizing the antimicrobial agents on the substrate reduces concerns regarding toxicity of the agents and minimizes the development of antimicrobial resistance, while presenting large antimicrobial agent concentrations at the site of action on the surface of the substrate. The combination of bacteriostatic activity with the active killing action of the membrane targeting antimicrobials further enhances the ability of these formulations to prevent microbial biofilm formation.

### DETAILED DESCRIPTION OF THE INVENTION

### I. Definitions

“Amino acid residue” and “peptide residue”, as used herein, refer to an amino acid or peptide molecule without the —OH of its carboxyl group (C-terminally linked) or one proton of its amino group (N-terminally linked). In general the abbreviations used herein for designating the amino acids and the protective groups are based on recommendations of the IUPAC-IUB Commission on Biochemical Nomenclature (see Biochemistry (1972) 11:1726-1732). Amino acid residues in peptides are abbreviated as follows: Alanine is Ala or A; Cysteine is Cys or C; Aspartic Acid is Asp or D; Glutamic Acid is Glu or E; Phenylalanine is Phe or F; Glycine is Gly or G; Histidine is His or H; Isoleucine is Ile or I; Lysine is Lys or K; Leucine is Leu or L; Methionine is Met or M; Asparagine is Asn or N; Proline is Pro or P; Glutamine is Gln or Q; Arginine is Arg or R; Serine is Ser or S; Threonine is Thr or T; Valine is Val or V; Tryptophan is Trp or W; and Tyrosine is Tyr or Y. Formylmethionine is abbreviated as fMet or Fm. By the term “residue” is meant a radical derived from the corresponding \ddot{γ}-amino acid by eliminating the OH portion of the carboxyl group and one of the protons of the \ddot{γ}-amino group. The term “amino acid side chain” is that part of an amino acid exclusive of the CH(NH₂)COOH backbone, as defined by K, D. Kopple, “Peptides and Amino Acids”, W. A. Benjamin Inc., New York and Amsterdam, 1966, pages 2 and 33; examples of such side chains of the common amino acids are —CH₂CH₂SCH₃ (the side chain of methionine), —CH₂(CH₃)—CH₂ CH₃ (the side chain of isoleucine), —CH₂CH(CH₃)₂ (the side chain of leucine) or —H (the side chain of glycine).

“Non-naturally occurring amino acid”, as used herein, refers to any amino acid that is not found in nature. Non-natural amino acids include any D-amino acids, amino acids with side chains that are not found in nature, and peptidomimetics. Examples of peptidomimetics include, but are not limited to, b-peptides, g-peptides, and d-peptides; oligomers having backbones which can adopt helical or sheet conformations, such as compounds having backbones utilizing bipyridine segments, compounds having backbones utilizing solvophobic interactions, compounds having backbones utilizing side chain interactions, compounds having backbones utilizing hydrogen bonding interactions, and compounds having backbones utilizing metal coordination. * All of the amino acids in the human body, except glycine, exist as the D and L forms. Nearly all of the amino acids occurring in nature are the L-forms. D-forms of the amino acids are not found in the proteins of higher organisms, but are present in some lower forms of life, such as in the cell walls of bacteria. They also are found in some antibiotics, among them, streptomycin, actinomycin, bacitracin, and tetracycline. These antibiotics can kill bacterial cells by interfering with the formation of proteins necessary for viability and reproduction. Non-naturally occurring amino acids also include residues, which have side chains that resist non-specific protein adsorption, which may be designed to enhance the presentation of the antimicrobial peptide in biological fluids, and/or polymerizable side chains, which enable the synthesis of polymer brushes using the non-natural amino acid residues within the peptides as monomeric units.

“Polypeptide”, “peptide”, and “oligopeptide” encompasses organic compounds composed of amino acids, whether natural, synthetic or mixtures thereof that are linked together chemically by peptide bonds. Peptides typically contain 3 or more amino acids, preferably more than 9 and less than 150, more preferably less than 100, and most preferably between 9 and 51 amino acids. The polypeptides can be “exogenous,” or “heterologous,” i.e. production of peptides within an organism or cell that are not native to that organism or cell, such as human polypeptide produced by a bacterial cell. Exogenous also refers to substances that are not native to the cells and are added to the cells, as compared to endogenous materials, which are produced by the cells. The peptide bond involves a single covalent link between the carboxyl group (oxygen-bearing carbon) of one amino acid and the amino nitrogen of a second amino acid. Small peptides with fewer than about ten constituent amino acids are typically called oligopeptides, and peptides with more than ten amino acids are termed polypeptides. Compounds with molecular weights of more than 10,000 Daltons (50-100 amino acids) are usually termed proteins.

“Antimicrobial” as used herein, refers to molecules that kill (i.e., bactericidal) or inhibit the growth of (i.e., bacteristatic) microorganisms including bacteria, yeast, fungi, mycoplasma, viruses or virus infected cells, cancerous cells, and/or protozoa.

“Antimicrobial peptide” (“AmP”), as used herein, refers to oligopeptides, polypeptides, or peptidomimetics that kill (i.e., are bactericidal) or inhibit the growth of (i.e., are bacteristatic) microorganisms including bacteria, yeast, fungi, mycoplasma, viruses or virus infected cells, and/or protozoa. Generally, antimicrobial peptides are cationic molecules with spatially separated hydrophobic and charged regions. Exemplary antimicrobial peptides include linear peptides that form an α-helical structure in membranes or peptides that form β-sheet structures, optionally stabilized with disulfide bridges in membranes. Representative antimicrobial peptides include, but are not limited to, cathelicidins, defensins, dermcidin, and more specifically magainin 2, protegrin, protegrin-1, melittin, 11-37, dermaseptin 01, cecropin, caerin, ovispirin, cecropin A melittin hybrid, and alamethicin, or hybrids or analogues of other AmPs. Naturally occurring antimicrobial peptides include peptides from vertebrates and non-vertebrates, including plants, humans, fungi, microbes, and insects.

“Adhesion”, as used herein, refers to the non-covalent or covalent attachment of a protein, cell, or other substance to a surface. The amount of adhered substance may be quantified by sonicating and/or rinsing the surface with an appropriate resuspension agent such as Tween or SDS, and quantifying the amount of substance resuspended.

“Bioactive agent” or “active agent” or “biomolecule”, used here synonymously, refers to any organic or inorganic therapeutic, prophylactic or diagnostic agent that actively or passively influences a biological system. For example, a bioactive agent can be an amino acid, antimicrobial peptide, immunoglobulin, an activating, signaling or signal amplifying molecule, including, but not limited to, a protein kinase, a cytokine, a chemokine, an interferon, tumor necrosis factor, growth factor, growth factor inhibitor, hormone, enzyme, receptor-targeting ligand, gene silencing agent, ambisense, antisense, an RNA, a living cell, cohesin, laminin, fibronectin, fibrinogen, osteocalcin, osteopontin, or osteoprotegerin. Bioactive agents can be proteins, glycoproteins, peptides, oligliopeptides, polypeptides, inorganic compounds, organometallic compounds, organic compounds or any synthetic or natural, chemical or biological compound.

“Non-fouling”, as used herein, means that the composition reduces or prevents the amount of adhesion of proteins, including blood proteins, plasma, cells, tissue and/or microbes to the substrate relative to the amount of adhesion to a reference polymer such as polyurethane. Preferably, a device surface will be substantially non-fouling in the presence of human blood. Preferably the amount of adhesion will be decreased 20%, 50%, 75%, 90%, 95%, or most preferably 99%, 99.5%, 99.9% relative to the reference polymer. Non-fouling activity with respect to protein, also referred to as “non-specific protein adsorption resistance” may be measured using an ELISA assay. For solutions containing only a single protein, protein adsorption can be measured by ELISA assay. The sample is first incubated in the protein solution, then rinsed to remove loosely adhered proteins. It is then exposed to a solution containing a calorimetrically labeled antigen to the specific protein and once again rinsed to remove loosely adhered material. Finally, the substrate is treated with solution to remove the antigen and the concentration of the antigen measured by UV-Vis spectroscopy. For mixed protein solutions, such as whole plasma, surface plasmon resonance (SPR) or optical waveguide lightmode spectroscopy (OWLS) can be utilized to measure surface protein adsorption without necessitating the use of individual antigens for each protein present in solution. Additionally, radiolabeled proteins may be quantified on the surface after adsorption from either one protein or complex mixtures. Non-fouling activity with respect to bacteria may be quantified by exposing treated substrates (and untreated controls) to between 1×10⁵-10⁷ CFU/ml of a given organism suspended in PBS or more complex media for 2 hours. The samples are then rinsed to remove loosely adherent cells, placed in fresh PBS, and then sonicated to re-suspend the adherent bacteria in solution. Serial dilutions of this supernatant solution can then be made, plated, and grown up over night to provide a quantitative measure of bacterial adhesion on the treated sample versus the control. Preferably at least a 1, 2, 3 or 4 log reduction in bacterial count occurs relative to colonization on a control. Similar adherence assays are known in the art for assessing platelet, cell, or other material adhesion to the surface.

“Biocompatibility” is the ability of a material to perform with an appropriate host response in a specific situation (Williams, D. F. Definitions in Biomaterials. In: Proceedings of a consensus Conference of the European Society for Biomaterials. Elsevier: Amsterdam, 1987).

“Biological fluids” are fluids produced by organisms containing proteins and/or cells, as well as fluids and excretions from microbes. This includes, but is not limited to, blood, saliva, urine, cerebrospinal fluid, tears, semen, and lymph, or any derivative thereof (e.g., serum, plasma).

“Brush”, or “Polymer Brush” as used herein synonymously, refers to a relatively high density of polymer chains stretched away from the polymer or polymers due to the volume-excluded effect. The polymer-chains are typically end-tethered to the substrate. In mixed brushes, two or more different polymers grafted to the same substrate constitute the brush.

“Branch” and “Branched tether,” are used interchangeably and refer to a polymer structure which originates from a single polymer chain but terminates in two or more polymer chains. The polymer in question may be a homopolymer or multicomponent copolymer. Branched tether polymer structure may be ordered or random, may be composed, in whole or in part, of non-fouling material, and may be utilized to immobilize one or more molecules of one or more bioactive agents. In one embodiment the branched tether is a dendrimer. A branched tether may be immobilized directly to a substrate or to a coating covering a substrate.

“Coupling agent”, as used herein, refers to any molecule or chemical substance which activates a chemical moiety, either on the bioactive agent or on the material to which it will be attached, to allow for formation of a covalent or non-covalent bond between the bioactive agent wherein the material does not remaining in the final composition after attachment.

“Cysteine”, as used herein, refers to the amino acid cysteine or a synthetic analogue thereof, wherein the analogue contains a free sulfhydryl group.

“Degradation products” are atoms, radicals, cations, anions, or molecules which are derived from a bioactive agent or composition and which are formed as the result of hydrolytic, oxidative, enzymatic, or other chemical processes over the course of 14, 30, 120, 365, or 1000 days.

“Density”, as used herein, refers to the mass of material, which without limitation may include non-fouling materials or bioactive agents, that is immobilized per surface area of substrate.

“Effective surface density”, as used herein, means the range of densities suitable to achieve an intended surface effect, which without limitation may be antimicrobial or non-fouling effect.

“Hydrophilic” refers to polymers, materials, or functional groups which generally associate with water. These materials include without limitation materials with hydroxyl, zwitterionic, carboxy, amino, amide, phosphate, hydrogen bond formers, and ether.

“Immobilization” or “immobilized”, as used herein, refers to a material or bioactive agent that is covalently attached directly or indirectly to a substrate. “Co-immobilization” refers to immobilization of two or more agents.

“In vivo stability” refers to materials which are not degraded in organism over a defined period of time.

“Non-degradable” or “stable”, as used herein synonymously, refers to material compositions that do not react within a biological environment either hydrolytically, reductively, enzymatically or oxidatively to cleave into smaller pieces. Preferably non-degradable materials retain >25%, >50%, >75%, >90%, >95%, or >99% of their original material properties such as surface contact angle, non-fouling, and/or bactericidal activity for a time of 7, 14, 30, 120, 365, or 1000 days in media, serum, or in vivo.

“Substrate”, as used herein, refers to the material on which a non-fouling coating is applied, or which is formed all or in part of non-fouling material, or on which the non-fouling and/or anti-microbial agents are immobilized.

“Membrane-targeting antimicrobial agent”, as used herein, refers to any antimicrobial agent that retains its bactericidal or bacteriostatic activity when immobilized on a substrate and can therefore be used to create an immobilized antimicrobial surface. In one embodiment, the membrane-targeting antimicrobial agent is an antimicrobial peptide, and in another embodiment it is a quaternary ammonium compound or polymer. “Immobilized bactericidal activity” as used herein, refers to the reduction in viable microorganisms including bacteria, yeast, fungi, mycoplasma, viruses or virus infected cells, and/or protozoa that contact the surface. For bacterial targets, bactericidal activity may be quantified as the reduction of viable bacteria based on the ASTM 2149 assay for immobilized antimicrobials, which may be scaled down for small samples as follows: an overnight culture of a target bacteria in a growth medium such as Cation Adjusted Mueller Hinton Broth, is diluted to approximately 1×10⁵ cfu/ml in pH 7.4 Phosphate Buffered Saline using a predetermined calibration between OD600 and cell density. A 0.5 cm² sample of immobilized antimicrobial surface is added to 0.75 ml of the bacterial suspension. The sample should be covered by the liquid and should be incubated at 37° C. with a sufficient amount of mixing that the solid surface is seen to rotate through the liquid. After 1 hour of incubation, serial dilutions of the bacterial suspension are plated on agar plates and allowed to grow overnight for quantifying the viable cell concentration. Preferably at least a 1, 2, 3 or 4 log reduction in bacterial count occurs relative to a control of bacteria in phosphate buffered saline (PBS) without a solid sample.

“Coating”, as used herein, refers to any temporary, semi-permanent or permanent layer, or layers, treating or covering a surface. The coating may be a chemical modification of the underlying substrate or may involve the addition of new materials to the surface of the substrate. It includes any increase in thickness to the substrate or change in surface chemical composition of the substrate. A coating can be a gas, vapor, liquid, paste, semi-solid or solid. In addition, a coating can be applied as a liquid and solidified into a solid coating.

“Undercoating,” as used herein, refers to any coating, combination of coatings, or functionalized layer covering an entire substrate surface or a portion thereof under an additional coating. In one embodiment, the undercoating is used to alter the properties of one or more subsequent coatings or layers.

“Top coating,” as used herein, refers to any coating, combination of coatings, or functionalized layer applied on top of a undercoating, another top coating or directly to a substrate surface. A top coating may or may not be the final coating applied to a substrate surface. In one embodiment a top coat is covalently attached to a primer undercoating. In another embodiment a top coating is encapsulated in a protective coating, which helps extend the top coatings storage life.

“Substantially Cytotoxic”, as used herein, refers to a composition that changes the metabolism, proliferation, or viability of mammalian cells that contact the surface of the composition. These may be quantified by the International Standard ISO 10993-5 which defines three main tests to assess the cytotoxicity of materials including the extract test, the direct contact test and the indirect contact test.

“Substantially hemocompatible”, as used herein, means that the composition is substantially non-hemolytic, in addition to being non-thrombogenic and non-immunogenic, as tested by appropriately selected assays for thrombosis, coagulation, and complement activation as described in ISO 10993-4.

“A substantially non-hemolytic surface”, as used herein, means that the composition does not lyse 50%, preferably 20%, more preferably 10%, even more preferably 5%, most preferably 1%, of human red blood cells when the following assay is applied: A stock of 10% washed pooled red blood cells (Rockland Immunochemicals Inc, Gilbertsville, Pa.) is diluted to 0.25% with a hemolysis buffer of 150 mM NaCl and 10 mM Tris at pH 7.0. A 0.5 cm² antimicrobial sample is incubated with 0.75 ml of 0.25% red blood cell suspension for 1 hour at 37° C. The solid sample is removed and cells spun down at 6000 g, the supernatant removed, and the OD414 measured on a spectrophotometer. Total hemolysis is defined by diluting 10% of washed pooled red blood cells to 0.25% in sterile deionized (DI) water and incubating for 1 hour at 37° C., and 0% hemolysis is defined using a suspension of 0.25% red blood cells in hemolysis buffer without a solid sample.

“Non-leaching” or “Substantially non-leaching”, as used herein synonymously, means that the compositions retains >50%, 75%, 90%, 95%, 99% of the immobilized bioactive agent over the course of 7, 14, 30, 90, 365, 1000 days. This can be assessed using radiolabeled active agent followed by implantation in a relevant biological environment.

“Substantially non-toxic”, as used herein, means a surface that is substantially non-hemolytic and substantially non-cytotoxic.

“Tether” or “tethering agent” or “Linkert”, as used herein synonymously, refers to any molecule, or set of molecules, or polymer used to covalently immobilize a bioactive agent on a material where the molecule remains as part of the final chemical composition. The tether can be either linear or branched with one or more sites for immobilizing bioactive agents. In one embodiment, the tether is greater than 3 angstroms in length. Optionally, the tether may be non-fouling or a zwitterionic polymer. The tether may be immobilized directly on the substrate or on a polymer, either of which may be non-fouling.

“Zwitterion” or “zwittterionic material” refers to macromolecule, material, or moiety possessing both cationic and anionic groups. In most cases, these charged groups are balanced, resulting in a material with zero net charge. Zwitterionic polymers may include both Polyampholyte (the charged groups on different monomer units) and polybetaine (polymers with the anionic and cationic groups on the same monomer unit). Examples of materials which are not zwitterionic include poly(ethylene glycol).

### II. Compositions

A. Substrates

The antimicrobial agents may be applied to, or coupled to, a variety of different substrates. Examples of suitable materials include, but are not limited to, metallic materials, ceramics, polymers, woven and non-woven fibers, inert materials such as silicon, and combinations thereof.

Suitable metallic materials include, but are not limited to, metals and alloys based on titanium (such as nitinol, nickel titanium alloys, thermo-memory alloy materials), stainless steel, tantalum, palladium, zirconium, niobium, molybdenum, nickel-chrome, or certain cobalt alloys including cobalt-chromium and cobalt-chromium-nickel alloys such as ELGILOY® and PHYNOX®.

Suitable ceramic materials include, but are not limited to, oxides, carbides, or nitrides of the transition elements such as titanium oxides, hafnium oxides, iridium oxides, chromium oxides, aluminum oxides, and zirconium oxides. Silicon based materials, such as silica, may also be used.

Suitable polymeric materials include, but are not limited to, polystyrene and substituted polystyrenes, polyethylene, polypropylene, poly(urethane)s, polyacrylates and polymethacrylates, polyacrylamides and polymethacrylamides, polyesters, polysiloxanes, polyethers, poly(orthoester), poly(carbonates), poly(hydroxyalkanoate)s, polyfluorocarbons, PEEK®, Teflon® (polytetrafluoroethylene, PTFE), silicones, epoxy resins, Kevlar®, Dacron® (a condensation polymer obtained from ethylene glycol and terephthalic acid), nylon, polyalkenes, phenolic resins, natural and synthetic elastomers, adhesives and sealants, polyolefins, polysulfones, polyacrylonitrile, biopolymers such as polysaccharides and natural latex, and combinations thereo. In one embodiment the substrate can be a medical grade polyurethane material (including aromatic and aliphatic and polyether or polycarbonate based polymers), such as the Thermedics™ Polymer Products provided by The Lubrizol Corporation which include Carbothane™ and Tecoflex™, blended with appropriate extrusion agents and plasticizers, the material preferably being already approved by the FDA or other appropriate regulatory agency for use in vivo.

The micro and nano structure of the substrate surface is important in order to maximize the surface area available for antimicrobial agent attachment. For metallic and ceramic substrates, increased surface area can be created through surface roughening, for example, by a random process such as plasma etching. Alternatively, the surface can be modified by controlled nano-patterning using photolithography. Polymeric substrates can also be roughened as with metallic and ceramic substrates. In addition, the surface area available for antimicrobial agent attachment on a polymeric substrate can be increased by controlling the morphology of the polymer itself, as discussed in more detail below.

The substrates may optionally contain a radiopaque additive, such as barium sulfate or bismuth to aid in radiographic imaging. Substrates may also contain radioactive materials, such as those implanted in the prostate for treatment of prostate cancer.

Substrates may be in the form of, or form part of, films, particles (nanoparticles, microparticles, or millimeter diameter beads), fibers (wound dressings, bandages, gauze, tape, pads, sponges, including woven and non-woven sponges and those designed specifically for dental or ophthalmic surgeries), or surgical, medical and/or dental instruments. In one embodiment the substrate is a vascularly inserted catheter such as a PICC, CVC, or hemodialysis catheter.

B. Membrane-Targeting Antimicrobial Agents

A variety of membrane-targeting antimicrobials have been discovered or created. This broad class of membrane destabilizing agents frequently has favorable selectivity between mammalian and bacterial membranes. Any peptide which exhibits antimicrobial properties when immobilized to a substrate can be used. Not all antimicrobial peptides have activity when immobilized, so it is essential to verify activity after immobilization. Methods and systems for generating peptides which exhibit antimicrobial activity when immobilized are described in U.S. Patent Application Publication No. 2006/0035281 by Stephanopoulos et al. For example, the pattern Q.EAG.L.K.K. (SEQ ID NO: 1) (where “.” is a wildcard, indicating that any amino acid will suffice at that position in the pattern) is present in over 90% of cecropins, an AmP common in insects. Computational tools, such as TEIRESIAS, can be used to produce libraries of peptides that exhibit antimicrobial activity. The peptides preferably show limited homology to naturally-occurring proteins but have strong bactericidal activity against several microbial species, including S. aureus and B. anthracis. The peptides can be synthesized using conventional methods, such as Fmoc chemistry. Once made, the designed proteins and peptides can be experimentally evaluated and tested for structure, function and stability, as required, using routine methods known to those skilled in the art. Suitable peptides are described in Wang, Z and G Wang, APD: the Antimicrobial Peptide Database, Nucleic Acids Research, 2004, Vol. 32, Database issue D590-D592 and include, but are not limited to, Cecropin-Melittin Hybrid (KWKLFKKIGAVLKVL-aminated) (SEQ ID NO: 2), Cecropin P1, Temporin A, D28, D51, dermaseptin, RIP, and combinations thereof. Optionally, additional amino acids may be attached to these peptides for a range of functions. In one embodiment, a cysteine or unnatural amino acid is appended on the sequence to be used in immobilization.

The use of D-amino acids in the synthesis of AmPs may also be advantageous because of their long term stability. Complex biological environments often contain many active enzymes that could cleave AmPs at residue specific locations, depending on sequence. This could result in enzymatic deactivation of an immobilized antimicrobial coating. One approach is to design agents that do not have sequences known to be protease targets. A preferable approach is the substitution of D-amino acid residues for the naturally occur L-amino residues in the synthesis of antimicrobial peptides to prevent enzymatic degradation. In a preferred embodiment, a peptide consisting entirely of D-amino acid residues is used. While this substitution will affect the chirality of the resulting peptide, it does not affect the immobilized activity of a model amphiphilic a-helical AmP, cecropin A melittin, and by extrapolation, other similar peptides. Addressing protease attack may be more important in immobilized applications than for peptides used systemically, given that it is desirable to present the immobilized peptides for the lifetime of the device, which may be permanent. The use of immobilization to prevent protease access to the peptides in addition to unnatural amino acids such as D-amino acids may further prevent degradation. Surprisingly, because of the non-chiral mechanism of AmPs, D-amino acid equivalent peptides retain bactericidal activity when immobilized on a surface.

Peptidomimetics, which exhibit antibacterial activity, may also be used. Peptidomimetics are molecules which mimic peptide structure. Peptidomimetics have general features analogous to their parent structures, polypeptides, such as amphiphilicity. Examples of peptidomimetic materials are described in Moore et al., Chem. Rev. 101(12), 3893-4012 (2001). The peptidomimetic materials can be classified into the following categories: α-peptides, β-peptides, γ-peptides, and δ-peptides. Copolymers of these peptides can also be used.

Examples of α-peptide peptidomimetics include, but are not limited to, N,N′-linked oligoureas, oligopyrrolinones, oxazolidin-2-ones, azatides and azapeptides.

Examples of β-peptides include, but are not limited to, β-peptide foldamers, α-aminoxy acids, sulfur-containing β-peptide analogues, and hydrazino peptides.

Examples of γ-peptides include, but are not limited to, γ-peptide foldamers, oligoureas, oligocarbamates, and phosphodiesters.

Examples of δ-peptides include, but are not limited to, alkene-based δ-amino acids and carbopeptoids, such as pyranose-based carbopeptoids and furanose-based carbopeptoids.

Another class of peptidomimetics includes oligomers having backbones which can adopt helical or sheet conformations. Example of such compounds include, but are not limited to, compounds having backbones utilizing bipyridine segments, compounds having backbones utilizing solvophobic interactions, compounds having backbones utilizing side chain interactions, compounds having backbones utilizing hydrogen bonding interactions, and compounds having backbones utilizing metal coordination.

Examples of compounds containing backbones utilizing bipyridine segments include, but are not limited to, oligo(pyridine-pyrimidines), oligo(pyridine-pyrimidines) with hydrazal linkers, and pyridine-pyridazines.

Examples of compounds containing backbones utilizing solvophobic interactions include, but are not limited to, oligoguanidines, aedamers (structures which take advantage of the stacking properties of aromatic electron donor-acceptor interactions of covalently linked subunits) such as oligomers containing 1,4,5,8-naphthalene-tetracarboxylic diimide rings and 1,5-dialkoxynaphthalene rings, and cyclophanes such as substituted N-benzyl phenylpyridinium cyclophanes.

Examples of compounds containing backbones utilizing side chain interactions include, but are not limited to, oligothiophenes such as olihothiophenes with chiral p-phenyl-oxazoline side chains, and oligo(m-phenylene-ethynylene)s.

Examples of compounds containing backbones utilizing hydrogen bonding interactions include, but are not limited to, aromatic amide backbones such as oligo(acylated 2,2′-bipyridine-3,3′-diamine)s and oligo(2,5-bis[2-aminophenyl]pyrazine)s, diaminopyridine backbones templated by cyanurate, and phenylene-pyridine-pyrimidine ethynylene backbones templated by isophthalic acid.

Examples of compounds containing backbones utilizing metal coordination include, but are not limited to, zinc bilinones, oligopyridines complexed with Co(II), Co(III), Cu(II), Ni(II), Pd(II), Cr(III), or Y(III), oligo(m-pheylene ethynylene)s containing metal-coordinating cyano groups, and hexapyrrins.

In one embodiment, the antimicrobial agent is the antimicrobial peptide Cecropin-Melittin Hybrid (KWKLFKKIGAVLKVL-aminated) (SEQ ID NO:2), D28 (FLGVVFKLASKVFPAVFGKV) (SEQ ID NO:3), DS51 (FLFRVASKVFPALIGKFKKK) (SEQ ID NO:4), MICL-1 (GIGKFLKKAKKFGKAFVKILKK-NH2) (SEQ ID NO:5), MICL-41 (RGLRRLGRKIAHGVKKYGPTVLRIIRIAG-NH2) (SEQ ID NO:6), or MICL-42 (GWKDWAKKAGGWLKKKGPGMAKAALKAAMQ-NH2) (SEQ ID NO:7). Optionally, additional amino acids may be attached to these peptides for a range of functions. In one embodiment, a cysteine or unnatural amino acid is appended on the sequence to be used in immobilization. It is preferable to use antimicrobial peptides that do not naturally occur in humans. Human AmPs, including defensins and LL-37, are involved in immune recruitment and signaling processes that may be unfavorable near the surface of a medical device.

Additional synthetic membrane targeting antimicrobials have been developed, Gabriel et al., Mat. Sci. & Eng. R, 57: 28-64 (2007). These agents are small molecules that can adopt AmP-like conformations and act to destabilize bacterial membranes in much the same way as AmPs.

C. Coating Formulations to Resist Non-Specific Protein Adsorption

The production of surfaces which resist non-specific protein adsorption is a key element in the development of biomedical materials, such as medical devices and implants. Such coatings limit the interactions between the implants and physiological fluids. In environments where fluids contain high concentrations of biological proteins, such as blood contacting applications, prevention of protein adsorption is essential in maintaining immobilized antimicrobial efficacy. Formation of a protein conditioning film on the surface of an immobilized antimicrobial coating may decrease the coating's ability to resist bacterial colonization. Different approaches can be adopted to create surfaces that resist non-specific protein adsorption, including the use of protein resistant tethers, protein resistant polymers or polymer brushes, or hydrogels covalently attached to the substrate.

Many different materials have been developed to resist non-specific protein adsorption. Chemistries utilized for this purpose include, but are not limited to: polyethers (polyethylene glycol in particular), polysaccharides such as dextran, more traditional hydrophilic polymers such as polyvinylpyrrolidone or hydroxyethyl-methacrylate, heparin, intramolecular zwitterions or mixed charge materials, and hydrogen bond accepting groups described by U.S. Pat. No. 7,276,286 to Chapman, et al. The ability of these materials in preventing protein adsorption varies greatly between the chemistries. The PEG/OEG-based polymers and zwitterionic polymers (e.g. PC-, SB-, CB-, and mixed charge-based polymers) possess the highest protein resistance among all the known polymers. However, CB- and SB-based polymers can be more stable than PEG/OEG and may retain high protein resistance for long term in vivo applications. In one embodiment, the polymer is a carboxybetaine acrylamide. In another embodiment, the polymer is an acrylamide. These acrylamide polymers may be used to increase stability.

To maximize the presentation of immobilized antimicrobial agents in a high protein concentration environment, a formulation should resist preferably greater than 50%, greater than 75%, greater than 90%, greater than 95%, greater than 99%, or greater than 99.9% of the adsorption of a monolayer of protein from solution, relative to an uncoated control. The protein adsorption from serum over 1 hour should be less than 20, less than 10, less than 5, less than 1, or less than 0.5 ng/cm². Most preferably total protein adsorption will be less than 1 ng/cm².

1. Antifouling Tethers

One option for an immobilized antimicrobial structure is to utilize an antifouling material as the tether between the antimicrobial and the surface. This can take the form of a chemical chain that is itself non-fouling, such as a linear PEG or polysaccharide. As described above, PEG chains degrade auto-oxidatively, likely preventing their use in creating coatings with long term antimicrobial efficacy. An alternative approach is to utilize a tether with a stable or hydrophobic backbone having many pedant nonfouling groups, such as intramolecular zwitterions or groups that are hydrogen bond acceptors but not donors. This allows both flexibility and protein adsorption resistance while providing stable attachment of the antimicrobial agent. In one embodiment the tether is a polymer brush. In a further embodiment the polymer brush possesses zwitterionic side chain moieties. In a further embodiment the zwitterionic moieties are a sulfobetaine or a carboxybetaine. This structure can also be utilized to create additional attachment sites for molecules of antimicrobial agent. While a linear tether has only a single attachment site at its end, a brush tether could have molecules attached on many of the pendant nonfouling groups or additional functional groups copolymerized within the brush, as well as optionally on the end. In one embodiment these non-fouling zwitterionic side-chains are carboxybetaine, sulfobetaine, or phosphorylcholine. In a further embodiment the additional reactive groups are amines or amino derivatives.

Some zwitterionic polymers including carboxybetaine have functional groups such as carboxylic acids to which biomolecules may be directly immobilized. This functionalization may be carried out without destroying the underlying non-fouling properties of the tether. An antibody-funtionalized polycarboxybetaine surfaces platform has been used to detect a target protein in blood plasma with high sensitivity (Vaisocherová et al, Anal. Chem., 80: 7894-7901, (2008)) Preferably <1%, 5%, 10%, 50% of pendant groups are functionalized to allow for retention of antifouling properties. However, this work focused on the use of specific binding antibodies on in vitro arrays, neither immobilized antimicrobials nor in vivo applications were considered.

Zwitterionic moieties including phosphorycholines, carboxybetaines, and sulfobetaines are biocompatible and nonfouling groups which are helpful for preparing biocompatible surfaces and materials. Phosphorylcholine is the hydrophilic head group of phospholipids which are the main component of the cellular membrane of red blood cells. The structure of carboxybetaine is similar to that of glycine betaine, which is one of the solutes vital to the osmotic regulation of living organisms. The structure of sulfobetaine is similar to that of 2-aminoethane sulfonic acid or taurine, which is present in high concentrations in animals and occurs in trace amounts in plants. The biomimetic structures of these zwitterionic groups make them nontoxic and biocompatible.

Polymers with zwitterionic moieties have non-fouling properties. For example, compositions containing less than 0.3 ng/cm² fibrinogen adsorption on polysulfobetaine methacrylate (SBMA) and polycarboxybetaine methacrylate (CBMA) are non-fouling. These zwitterionic polymer-grafted surfaces are highly resistant to nonspecific protein adsorption from plasma and serum, bacterial adhesion, biofilm formation, and platelet adhesion. Polycarboxybetaine polymers also exhibit anticoagulant properties. Most surfaces can not resist platelet attachment, which may sequentially induce thrombosis on the surfaces, unless the fibrinogen adsorption is less than 5-10 ng/cm². Carboxybetaine polymers have unique dual functionalities—they have abundant functional groups for convenient biomolecule immobilization and still maintain high resistance to non-specific protein and cell adhesion.

The compositions may also include a linker which is typically derived from a molecule having two or more functional groups capable of forming a covalent bond to another species. Typically, the two or more functional groups are located at the ends of the linker; however, one or more of the functional groups may be located at a position on the linker other than the ends. The linker can be a single atom, e.g., a sulfur, carbon, oxygen, or nitrogen atom, two or more atoms, such as an amide linker, or as large as an oligomer or polymer. The linker may contain one or more heteroatoms within the linker. Optionally, no linker is used, with the non-fouling group covalently attached directly to the amino antimicrobial agent. Optionally, a linker has pendant groups that are non-fouling, such as zwitterions, with a single antimicrobial immobilized on the end of the linker. Alternatively, no linker is used and the antimicrobial agent is bound to the substrate or a copolymer containing both non-fouling moieties and other reactive moieties through direct reaction of a group within the antimicrobial with a surface group or alternate reactive group.

Heterobifunctional crosslinking agents can also be utilized to create the linker. In one embodiment this crosslinking agent is Sulfo-GMBS. Other heterobifunctional crosslinking agents include, but are not limited to: −[a-maleimidoacetoxy]-succinimide ester (AMAS), N-β-Maleimidopropionic acid (BMPA), N—(R-Maleimidopropionic acid) hydrazide TFA (BMPH), N—R (Maleimidopropyloxy) succinimide ester (BMPS), N-£-Maleimidocaproic acid (EMCA), N-[e-maleimidocaproic acid]hydrazide (EMCH), N-(E-Maleimidocaproyloxy) sulfosuccinimide ester (EMCS), N—K-Maleimidoundecanoic acid (KMUA), N—(K-Maleimidoundecanoic acid) hydrazide (KMUH), LC-SMSS, m-Maleimidobenzoyl-N hydroxysuccinimide ester (MBS), 4-N-Maleimidomethyl)cyclohexane-1-carboxylhydrazide HCl (M2C2H), 4-(4-N-Maleimidophenyl) butyric acid hydrazide HCl (MPBH), N-succinimidyl S-acetylthioacetate (SATA), N-succinimidyl-S-acetylthiopropionate (SATP), and succinimidyl-4-(N-maleimidomethyl)cyclohexane-1-carboxylate (SMCC).

2. Antifouling Tethers not Covalently Coupled to Antimicrobial Agents

To create surfaces resistant to non-specific protein adsorption, antifouling moieties, which are not tethered to the antimicrobial agent, can be immobilized on the substrate. In this case, the antifouling polymer or moiety is not a tether but acts as a protein adsorption resistant agent. The non-fouling polymers described in the section above can be used for this purpose. This side-by-side incorporation of protein adsorption resistant materials can also be utilized in a polymer brush with some repeat units allowing for antimicrobial agent attachment while others provided pendant moieties that resist non-specific protein adsorption. These structures can be arranged randomly, with protein adsorption resistant and antimicrobial units interspersed, or as a block copolymer with protein adsorption resistant and antimicrobial repeat units segregated. In one embodiment, the antimicrobial units are subsequently attached to a functional moiety interspersed between protein resistant units. In one embodiment this functional moiety is an amine group. In one embodiment the protein adsorption resistant block connects the antimicrobial block to the substrate. In one embodiment the protein resistant block is composed of a polymer brush with pendant protein resistant moieties and the other block is composed of a polymer brush with pendant functional groups for subsequent attachment of membrane targeting antimicrobial agents. In one embodiment the protein resistant moieties are zwitterionic groups. In one embodiment these zwitterionic groups are carboxybetaine or sulfobetaine. In one embodiment the functional block is composed of polymerized aminoethylethacrylate. In another embodiment the antimicrobial block connects the protein adsorption resistant block to the substrate. Blocks can also be alternated.

3. Hydrogels

Hydrogels can be used as protein adsorption resistant coatings on the substrate, on other coatings or can be used as the substrate itself. In one embodiment, antimicrobial agents can be immobilized on the surface of the hydrogel, which is covalently or non-covalently immobilized on the surface of the substrate, provided the composition is non-leaching. In a preferred embodiment, the hydrogel is covalently bound to the substrate. Hydrogels are three-dimensional, hydrophilic, polymeric networks capable of imbibing large amounts of water or biological fluids (Peppas et al. Eur. J. Pharm. Biopharm. 2000, 50, 27-46). These networks are composed of homopolymers or copolymers, and are insoluble due to the presence of chemical crosslinks or physical crosslinks, such as entanglements or crystallites. Hydrogels can be classified as neutral or ionic, based on the nature of the side groups. In addition, they can be amorphous, semicrystalline, hydrogen-bonded structures, supermolecular structures and hydrocolloidal aggregates (Peppas, N. A. Hydrogels. In: Biomaterials science: an introduction to materials in medicine; Ratner, B. D., Hoffman, A. S., Schoen, F. J., Lemons, J. E., Eds; Academic Press, 1996, pp. 60-64; Peppas et al. Eur. J. Pharm. Biopharm. 2000, 50, 27-46). Hydrogels can be prepared from synthetic or natural monomers or polymers.

Medical devices can be coated with hydrogels using a variety of techniques, examples of which include spraying, dipping, blade coating, spin coating and brush coating. A small quantity of gel solution (e.g., in the microliter range) can be used to treat a surface area of 1 cm². The amount of gel solution per unit area and the corresponding coating solution concentration and application rate can be readily determined for any particular application.

Hydrogels can be prepared from synthetic polymers such as poly(acrylic acid) and its derivatives [e.g. poly(hydroxyethyl methaerylate) (pHEMA)], poly(N-isopropylacrylamide), poly(ethylene glycol) (PEG) and its copolymers and poly(vinyl alcohol) (PVA), among others (Bell, C. L.; Peppas, N. A. Adv. Polym. Sci. 1995, 122, 125-175; Peppas et al. Eur. J. Pharm. Biopharm. 2000, 50, 27-46; Lee, K. Y.; Mooney, D. J. Chem. Rev, 2001, 101, 1869-1879.). Hydrogels prepared from many synthetic polymers are non-degradable in physiologic conditions. In addition, hydrogels composed of synthetic polymers which tend to degrade as homopolymers can be made to resist degradation through control of the gel cross-link density. Hydrogels can also be prepared from natural polymers including, but not limited to, polysaccharides, proteins, and peptides. These networks are in general degraded in physiological conditions by chemical or enzymatic means. In one embodiment the hydrogel is formed of a sulfobetaine or carboxybetaine monomer and other appropriate monomers. Bioactive agents can then be immobilized on the hydrogel as described for tethers.

In one embodiment, the hydrogel is non-degradable under relevant in vitro and in vivo conditions. Stable hydrogel coatings are necessary for certain applications including central venous catheters coating, heart valves, pacemakers, stents coatings, capture of biological fluids as in the case of diapers and sanitary napkins, wound dressing, medical implants (e.g. breast implants and artificial cartilage), medical electrodes and contact lenses. In other cases, hydrogel degradation may be a preferential approach such as in tissue engineering constructs, sustained release drug delivery systems and topical drug delivery, specifically in the case of iontophoresis.

D. Other Active Agents

In addition to the antimicrobial agents, one or more therapeutic, prophylactic or diagnostic agents, which can be peptides, proteins, nucleic acids (e.g., DNA, RNA, etc.), or small organic or inorganic molecules, may be coupled to the substrate. In one embodiment, the substrate includes a bioactive agent which is released independently of the immobilized antimicrobial agents.

For example, agents which inhibit encapsulation, scarring, and/or cell proliferation may be immobilized with the antimicrobial agent on the substrate. Other examples of bioactive agents include, but are not limited to, antiproliferative, cytostatic or cytotoxic chemotherapeutic agents, antimicrobial agents, anti-inflammatories, growth factors, antithrombotics and anticoagulants such as heparin, and cell adhesion peptides (including RGD)) For blood-contacting application the combination of antithrombotic agents (such as heparin) and antimicrobial agents is particularly advantageous. For orthopedic applications the combination of bone growth or adhesion agents such as RGD peptide or hydroxyapatite with antimicrobial agents is particularly advantageous to improve integration and prevent loosening, while also reducing colonization. Bone morphogenic proteins (including, but not limited to, BMP2) and their analogues or mimetics are useful for orthopedic applications.

In another embodiment, one or more agents are tethered to the substrate using a hydrolyzable linkage so that the agent is slowly released from the substrate, for example, at the site of implantation or insertion of a medical device.

Alternatively, one or more agents are non-covalently associated with the surface. For example, one or more agents can be entrapped within a hydrogel material and released by diffusion and/or degradation of the hydrogel material. In one embodiment, the agents could be entrapped in a polymer layer applied to the substrate prior to functionalization with covalently immobilized membrane targeting antimicrobials and could then diffuse out through the polymer layer and the membrane targeting antimicrobial layer. In another embodiment multiple layers could be utilized to control drug release rate, dosing, and phasing of multiple drugs. In still another embodiment, one or more active agent can be incorporated into the substrate, for example, dispersed, homogeneously or heterogeneously, within a polymeric substrate.

### III. Methods for Immobilizing Membrane Targeting Antimicrobial Agents

The membrane-targeting antimicrobial agents can be applied to, immobilized on, or incorporated into or onto a substrate using a variety of covalent procedures known in the art. Coupling may be performed through direct reaction, use of a coupling agent, and/or use of a tethering agent.

Suitable covalent procedures include, but are not limited to, grafting a polymer to or from, or coating a polymer on the surface of a substrate to install reactive functional groups for coupling of the antimicrobial agents and direct attachment of the antimicrobial agents to the substrate surface. In one embodiment, the coupling reaction between an antimicrobial agent and the substrate involves a tethering group placed in the antimicrobial. The tethering group may be a thiol or vinyl, preferably at the antimicrobial agent terminal end, such that the antimicrobial agent is directly attached, with a specific orientation, to the polymeric coating on the surface of the medical device. In another embodiment a short tether molecule reacts preferentially with a pendant group in the polymer coating at one reactive site and reacts preferentially with a functional group at the terminal end of an antimicrobial agent, as in the case where a hydroxyl or amine acts as the pendant group in the polymer coating, methacrylic anhydride, maleic anhydride, or a vinyl or allyl halide acts as the short molecular tether and a thiol is present at the terminal end of the antimicrobial agent.

A. Means for Surface Attachment of Membrane Active Anti-Microbials

1. Direct Attachment to Surface

AmPs can be synthesized directly from a polymer surface, however this subjects the substrate surface to harsh chemical synthesis conditions which may affect the properties of the underlying material.

An alternative approach is to synthesize antimicrobial peptides, or other membrane targeting antimicrobial agents, in a batch process and then covalently couple the fully formed antimicrobial agent to the device. In one approach, the antimicrobial agent may be covalently attached to polymer chains throughout the bulk of the material making up a device. However, it is preferred to immobilize the antimicrobial agent on the surface of the device to reduce the mass and therefore cost of agent used, while also preventing an adverse effect on the bulk physical properties of the substrate material.

2. Tethers, Linkers, and Spacers

Both the structure and chemical composition of the molecules binding antimicrobial agents to the substrate have great influence on the antimicrobial efficacy of a composition. Tethers, linkers and spacers can be utilized both for attachment of antimicrobial agents to substrates and/or attachment of the agents to polymer films coated on substrates. The tether composition can be varied according to the surface chemistry of the substrate or the polymer covalently attached to, or coated onto, the substrate. Tether length can be varied to optimize antimicrobial agent interaction with bacteria encountering the surface and to maximize the anti-fouling properties of the surface. It may be desirable to have short tethers when producing covalently tethered antimicrobial surfaces to reduce cost.

Many membrane targeting antimicrobial agents, AmPs in particular, require the cooperative action of multiple molecules to kill microbes. The use of brushed or branched polymer tethers may increase the density and cooperativity of AmPs immobilized thereto. Increased cooperativity, or multivalency, may improve the antimicrobial activity relative to a composition having only one AmP immobilized per tether. In one embodiment the tether is a branched polymer brush possessing both non-fouling moieties and reactive moieties for coupling of membrane targeting antimicrobials. In a further embodiment the non-fouling moieties are zwitterionic in nature and the reactive moieties are chosen from the group of amines (and their derivatives), amides (and their derivatives), carboxylic acids (and their derivatives), azides, maleimides, or alkynes.

The use of flexible ether or ester based tethers such as PEG to increase mobility of bound anti-microbials may improve activity. However, polyethers, PEG in particular, are known to autoxidize in the presence of oxygen and transition metals. These environmental parameters are frequently encountered in vivo making polyether materials unsuitable for formulations intended to provide extended antimicrobial protection in this environment. In addition, esters are known to hydrolyze under physiological conditions, accelerated under either acidic of basic conditions, and are therefore commonly used as biodegradable materials (e.g., PLLA). The stability of the tethering structures used is critical for extended efficacy of the antimicrobial agent. In particular, a study synthesizing an AmP on a linear PEG chain on a polymer resin found that the AmP and a portion of the PEG was being cleaved and released into solution (Appendini et al, J. App. Polymer Sci., 81(3): 609-616 (2001)). Cleavage of the PEG was confirmed by measuring the molecular weight of the antimicrobial fraction in solution.

It would be desirable to use more stable tethering strategies (e.g., shorter chains, crosslinked chains, or non-cleavable chains) to lengthen the duration of activity and reduce toxicity concerns from agents being released into the body. In addition, coating compositions must remain in place on the surface of the substrate and not dissolve under biological conditions. U.S. Patent Application Publication No. 20070048249 discloses antimicrobial polymer coatings that, while effective in preventing bacterial colonization, dissolve in aqueous media. These formulations will only provide protection of the surface for a limited period.

To ensure long term formulation efficacy, the stability of the coatings should be assessed both in vitro and in vivo. In vitro samples can be incubated at 20-25° C. or 37° C. in a fluid relevant to the desired application (PBS, serum, whole blood, cerebrospinal fluid, etc.). Preferably the incubation time is 1 hour, 12 hours, 1 day, 7 days, 30 days, or 365 days. Following incubation, samples should be analyzed for coating thickness, by profilometry or ellipsometry, to ensure no bulk coating loss. Coatings should also be analyzed chemically, by XPS, to ensure retention of immobilized antimicrobial agents. Alternately, radio labeled antimicrobial agents can be utilized in the formulation, preferably 125I, and isotope counts measured following incubation to determine active agent retention. Finally, coatings can then be tested for biological activity through bacterial challenge. Similar testing can be performed on samples following in vivo implantation with the surface exposed to an applicable body compartment. Alternatively, accelerated in vitro degradation studies can be performed in appropriate media at elevated temperature. Specific chemistries that provide for both flexible and non-degradable tethers should utilize chemical bonds that are able to resist the hydrolytic, enzymatic, and oxidative environments encountered in vivo. Chemical linkages such as ethers, esters, thioethers and thioesters do not generally fulfill these criteria and are better replaced by linkages which do not degrade enzymatically, hydrolytically, or oxidatizely. In one embodiment this stable linkage is an amide. In another embodiment this stable linkage is a carbon-carbon bond. In a further embodiment the carbon-carbon chain possesses non-fouling side moieties and, optionally, reactive side chain moieties for immobilization of membrane targeting antimicrobial agents. In a further embodiment the non-fouling side chain moieties are zwitterionic groups connected to the carbon-carbon backbone through amide moieties and the optional reactive groups, also connected to the carbon-carbon backbone through amide moieties, are selected from the group of amines (and their derivatives), amides (and their derivatives), carboxylic acids (and their derivatives), azides, maleimides, or alkynes. In a further embodiment the zwitterionic groups are caboxybetaines, sulfobetaines, phosphorylcholines, or a combination thereof.

3. Dendrimers and Branched Polymers

Dendrimers and branched polymers provide an alternate approach for presenting surface bound antimicrobials in a manner that allows for cooperative action. Dendrimers have been demonstrated by many investigators to allow for multivalent interaction of ligands with cells or bacteria (Smith et al., Topics in Current Chem., 210: 183-227 (2000)). In one embodiment the dendrimeric structure is polyamidoamide (PAMAM). In another embodiment the branched polymeric structure would be polyethylene imine (PEI). These structures can provide a densely functional platform with high antimicrobial activity without necessitating long or flexible tethers for each molecule of immobilized agent. In addition, as previously demonstrated in US Patent Application 2007004394, compositions are selected such that the antimicrobial agent retains the correct orientation when presented on the surface have increased biological activity. By combining orientation and structures that provide for multivalent interaction, efficacy can by further enhanced. In addition, long flexible tethers can be combined with dendrimeric or branched structures to further enhance efficacy through density and cooperativity. In one embodiment the denrimeric polymer is a non-fouling polymer. In a further embodiment the nonfouling properties are imparted on the polymer by zwitterionic side chains. In yet a further embodiment the zwitterions are carboxybetaines, sulfobetaines, phosphorylcholines, or a combination thereof.

B. Covalent Procedures for Coupling Membrane Targeting Antimicrobials to a Substrate

1. Attachment of the Peptide to the Substrate Surface or Tether

The chemistry used to couple the antimicrobial agent to the substrate depends on the chemical composition of the substrate surface. The substrate surface can be treated in a variety of ways known in the art to introduce the desired functional group(s). Surface modification can be accomplished through gas-phase techniques including, but not limited to, plasma, corona discharge, flame treatment, UV/ozone, UV or ozone alone, electrochemistry, or wet chemistry including, but not limited to, aminolysis, hydrolysis, reduction, oxidation activation of alcohol chain ends with tosyl chloride and subsequent chemistry, graft copolymerisation of compounds with vinyl functionality by chemical initiation, and ion beam treatment in the presence of vinyl monomers. For example, the substrate surface can be treated with a plasma, microwave, and/or corona source to introduce hydroxyl, amine, and/or carboxylic acid groups to the substrate surface.

The coupling of the peptide to the substrate may also be accomplished using a tether. The tether may be bifunctional with one group reacting with a functional site on the surface, and the other group reacting with a specific site on the antimicrobial agent to provide oriented tethering. In one embodiment, the tether may have terminal functionalities that react with surface-amine and antimicrobial agent sulfhydryl groups. These tethers may contain a variable number of atoms. Examples of tethers include, but are not limited to, N-Succinimidyl 3-(2-pyridyldithio)propionate (SPDP, 3- and 7-atom spacer), long-chain-SPDP (12-atom spacer), (Succinimidyloxycarbonyl-□-methyl-2-(2-pyridyldithio) toluene) (SMPT, 8-atom spacer), Succinimidyl-4-(N-maleimidomethyl)cyclohexane-1-carboxylate) (SMCC, 11-atom spacer) and Sulfosuccinimidyl-4-(N-maleimidomethyl)cyclohexane-1-carboxylate, (sulfo-SMCC, 11-atom spacer), m-Maleimidobenzoyl-N hydroxysucecinimide ester (MBS, 9-atom spacer), N-(γ-maleimidobutyryloxy)succinimide ester (GMBS, 8-atom spacer), N-(γ-maleimidobutyryloxy) sulfosuccinimide ester (sulfo-GMBS, 8-atom spacer), Succinimidyl 6-((iodoacetyl)amino) hexanoate (SIAX, 9-atom spacer), Succinimidyl 6-(6-(((4-iodoacetyl)amino)hexanoyl)amino)hexanoate (SIAXX, 16-atom spacer), and p-nitrophenyl iodoacetate (NPIA, 2-atom spacer). One ordinarily skilled in the art also will recognize that a number of other coupling agents, with different number of atoms, may be used. In a preferential embodiment, the succinimide group of sulfo-GMBS is reacted with the amine groups from the substrate surface. In a subsequent step, the terminal maleimide group from sulfo-GMBS is reacted with sulfhydryl groups from the antimicrobial agent.

The coupling agent could also target non-natural amino acids placed in specific locations in the antimicrobial peptide. The non-natural amino acids may optionally be placed in only one position in the peptide to create uniform peptide orientation when the peptide is tethered. Non-natural amino acids generally include at least one functional group that can react with a corresponding functional group on a coupling agent or tether or directly in the polymer backbone and thereby create a point of attachment. The non-naturally occurring amino acids include at least one of the following functionalities: amine including primary, secondary and tertiary amine groups, hydroxyl, sulfhydryl, carboxyl, and phenol, zwitterionic groups and their derivatives, including carboxybetaine, sulfobetaine, phosphorylcholine groups. Coupling agents include compounds with any functional groups that will react with above non-natural amino acids and thereby create a covalent bond. For example, such coupling agents include 1-ethyl-3-3 dimethylaminopropylcarbodmiide (EDC), dicyclohexylcarbomide (DDC), glutaraldehyde, cyanogen bromide or N-hydroxysuccinimide.

In certain embodiments, the free amine groups of the antimicrobial peptide are attached to a surface containing reactive hydroxyl groups, in a non-oriented way. As an example, N,N′-Carbonyldiimidazole (CDI) can activate the hydroxyl groups of the surface with the concomitant formation of an imidazole carbamate. This reaction must take place in nonaqueous environments (e.g., acetone, dimethyl sulfoxide (DMSO), tetrahydrofuran (THF), dimethylformamide (DMF)) with less than 1% water due to the rapid breakdown of CDI by hydrolysis. Finally, the activated surface can react with an amine-containing membrane targeting antimicrobial agent solubilized in a buffer with pH between 7 and 10 (Ferreira et al., J. Molecular Catalysis B: Enzymatic 2003, 21, 189-199).

In other embodiments, the free amine groups of the antimicrobial agent are attached to a surface containing reactive amine groups. There is no control in peptide orientation using this chemistry. Tethers such as dithiobis(succinimidylpropionate) (DSP, 8-atom spacer), disuccinimidyl suberate (DSS, 8-atom spacer), glutaraldehyde (4-atom spacer), Bis[2-(succinimidyloxycarbonyloxy)ethyl]sulfone (BSOCOES, 9-atom spacer) and others that one skilled in the art also will recognize, can be used for this purpose.

In another embodiment, the tether may contain identical functional groups at each end that react with functional groups on the substrate and the peptide. For example, a homobifunctional tether is first reacted with a thiol surface in aqueous solution (for example PBS pH 7.4) and then in a second step the thiol containing antimicrobial agent is coupled to the tether. Examples of homobifunctional sulfhydryl-reactive tethers include, but are not limited to, 1,4-Di-[3′-2′-pyridyldithio)propion-amido]butane (DPDPB, 16-atom spacer) and Bismaleimidohexane (BMH, 14-atom spacer).

The choice of concentration of the tether utilized for activity will vary as a function of the volume, agent and substrate chosen for a given application, as will be appreciated by one skilled in the art.

Following immobilization, the surface may be washed with water or phosphate buffer saline or other buffer to remove unreacted antimicrobial agent and solvent. The buffer may contain small amounts of a surfactant (e.g., Sodium dodecyl sulfate, Tween®, Triton®) to facilitate the removal of the antimicrobial agent that is not covalently immobilized.

2. Grafting Polymers to a Substrate

In the preferred embodiment, a polymer is grafted onto a substrate and the membrane targeting antimicrobial agent is covalently coupled to the polymer. This polymer can be a homopolymer or copolymer possessing non-fouling properties. The polymer is chosen based on the desired functional group to be used to couple the membrane targeting antimicrobial agent to the substrate. Examples of suitable functional groups on the polymer include, but are not limited to, amines, carboxylic acids, epoxides, and aldehydes. In another embodiment, reactive monomers containing the desired functional groups can be polymerized on a substrate using techniques such as chemical vapor deposition (CVD).

Polymer Growth in Solution or from the Surface of the Substrate

Polymers can be grafted to a substrate using a variety of techniques known in the art. For example, the polymer can be grown in solution and then coupled to the surface of the substrate, known as grafting to. Alternatively, the polymer can be grown from the substrate surface, i.e. grafting from. The polymer can be grown in solution or from the substrate using a variety of polymerization techniques including, but not limited to, free radical polymerization, living polymerization, condensation polymerization, anionic polymerization, cationic polymerization, acyclic diene metathesis, polymerization, ring opening metathesis polymerization, and enzymatic polymerization. Polymer preparation methods include, but are not limited to, solution polymerization, bulk polymerization, emulsion polymerization, vapor phase polymerization. Polymers grown from the substrate can also be prepared using dendrimer synthesis. Examples of free-radical polymerization include spontaneous UV polymerization; type 1 or type 2 UV initiated polymerization; thermal initiated polymerization using a thermal initiator, such as AIBN; or redox-pair initiated polymerization. In the case of the polymers grown from the surface of the substrate, the surface is typically functionalized with the same moiety used for polymerization (e.g., vinyl groups for free radical polymerization).

Suitable polymers include, but are not limited to, poly(lactone), poly(anhydride), poly(urethane), poly(orthoester), poly(ethers), poly(esters), poly(phosphazine), poly(ether ester)s, poly(amino acids), synthetic poly(amino acids), poly(carbonates), poly(hydroxyalkanoate)s, polysaccharides, cellulosic polymers, proteins, such as zein, modified zein, casein, gelatin, gluten, serum albumin, collagen, actin, -fetoprotein, globulin, macroglobulin, cohesin, laminin, fibronectin, fibrinogen, osteocalcin, osteopontin, osteoprotegerin, and blends and copolymers thereof. These suitable polymers may possess non-fouling side moieties. In one embodiment, the polymer will possess zwitterionic side moieties to impart non-fouling properties on the composition.

For example, polymer brushes, combs, copolymers, and hydrogels can be formed by traditional synthetic means including, but not limited to, free radical polymerization, ionic polymerization, and ATRP. In addition, tethers or brush molecules can be formed either by grafting from the substrate, where the non-fouling structure in created in situ on the surface or by grafting to, where the non-fouling molecule is formed in solution and subsequently tethered to the substrate. In the case of grafting to, attachment of antimicrobial molecules to the nonfouling structure can occur either before or after the nonfouling structure is attached to the surface. Grafting-from approaches necessitate attachment of antimicrobials subsequent to surface attachment. Alternatively, antimicrobial agents may serve as terminators for the graft polymerization.

In another embodiment, when the antimicrobial agent is an AmP, the protein adsorption resistant structure can be incorporated into the peptide molecule itself during synthesis.

Alternately, a brush style structure could be formed from these peptides by incorporating a traditionally polymerizable unit at the end of the protein adsorption resistant section of the peptide. Examples of this moiety could include, but are not limited to, unsaturated hydrocarbon groups, amides, acrylates, methacrylates, and epoxides. Subsequent polymerization and substrate attachment, again either grafting from or to, would present a structure with a hydrophobic backbone with pedant antimicrobial molecules attached through protein adsorption resistant chains. This technique has the advantages that the linker is non-degradable and manufacturing hurdles would be greatly reduced.

In one embodiment, a cysteine-incorporating Cecropin-Melittin hybrid peptide (KWKLFKKIGAVLKVLC-NH₂) (SEQ ID NO: 8), KWKLFKKIGAVLKVLC-aminated (SEQ ID NO:9), with a single point of attachment at the cysteine (C), was immobilized on aminated polymer brushes coupled to a substrate. The polymer brushes were prepared by polymerizing the brush monomer aminoethyl methacrylate in the presence of a vinyl presenting substrate. The peptide was immobilized using sulfo-GMBS chemistry.

Polymer brushes can also be attached to materials such as silicone or polyurethane, which are commonly used to make medical devices. As described above, the growth of polymer brushes typically requires the presence of vinyl moieties on the substrate. In order to introduce vinyl groups onto the surface of silicone substrates, the silicone can be treated with a pure oxygen plasma followed by emersion in ethanol to create a surface that is purely hydroxyl in nature. Following hydroxylation, the surface can be exposed to an evaporated vinyl silane, such as trichlorovinyl silane or trimethoxy-vinyl silane. The vinylated substrate can then be used to attach brush polymers. Silicone polymers can also be treated with triflic acid to introduce SiH groups which can be subsequently utilized to attach silicone chains containing appropriate functional groups to the surface. Polyurethane substrates can be treated using a plasma treatment with CO₂, O₂, and ammonia. The resulting hydroxyl and/or amine groups can be acrylated to form vinyl moieties on the surface followed by tethering of the polymer brushes. Alternately, amine functionalities can be introduced on the surface of a polyurethane substrate by treatment with a di-amino molecule such as hexamethyldiamine through aminolysis. Semi- and fully interpenetrating polymer networks can be used to introduce a polymer with amino groups into a polyurethane substrate.

C. Polymer Microstructures

The maximum possible surface loading of membrane active immobilized antimicrobial can be increased through the creation of microstructure on the substrate surface. For polymeric substrates, including hydrogel networks, this surface morphology can be created through appropriate polymer structural design, such as dendrimers and brush copolymers. One example of this is the growth of surface tethered dedrimeric polymers. Poly(amidoamine) (PAMAM) dendrimer can be grown from an amine presenting surface through alternating reactions of methyl acrylate and ethylene diamine (Nguyen et al. Langmuir, 2006, 22, 7825-7832). Each generation of dendrimer added effectively doubles the number of sites available for antimicrobial attachment. In addition, when synthesis is terminated after an amination step, the resulting material is an amine presenting polymer that may behave as an anti-fouling hydrogel, similar to poly(ethylene glycol) (Champman et al. Langmuir, 2001, 17, 1225-1233). In one embodiment, a branched zwitterionic polymer or copolymer can be utilized to both impart non-fouling properties on the composition and provide multivalent sites for bioactive molecule attachment.

Another example of tailoring polymer microstructure to increase antimicrobial agent surface loading is the growth of polymer brushes from the substrate surface. These are polymer chains tethered at one end to the substrate but extending from the substrate into the surrounding medium. This approach creates many additional sites for attachment of antimicrobial agents, the number of which depends on the molecular weight of the brush polymer. One such system is brush growth of poly(methyl acrylate) (PMA). In another embodiment the brush can be zwitterionic in nature. Following polymerization of even moderate molecular weight PMA the material can be functionalized, leading to the surface presentation of orders of magnitude more immobilized antimicrobial agent than that possible through direct surface attachment.

### IV. Methods of Use

The materials described above may be in the form of a medical device to which the antimicrobial agent is applied as a coating or which is formed of the antimicrobial mixed with substrate. Suitable devices include, but are not limited to, surgical, medical or dental instruments, ophthalmic devices, wound treatments (bandages, sutures, cell scaffolds, bone cements, particles), appliances, implants, scaffolding, suturing material, valves, pacemaker, stents, catheters, rods, implants, fracture fixation devices, pumps, tubing, wiring, electrodes, contraceptive devices, feminine hygiene products, endoscopes, wound dressings and other devices, which come into contact with tissue, especially human tissue, cells or fluids.

A. Fibrous and Particulate Materials

In one embodiment, the compositions are immobilized on a fibrous material, or are incorporated into a fibrous material or a coating on a fibrous material. These include wound dressings, bandages, gauze, tape, pads, sponges, including woven and non-woven sponges and those designed specifically for dental or ophthalmic surgeries (See, e.g., U.S. Pat. Nos. 4,098,728; 4,211,227; 4,636,208; 5,180,375; and 6,711,879), paper or polymeric materials used as surgical drapes and garments, disposable diapers, tapes, bandages, feminine products, sutures, and other fibrous materials such as gauze, pads, wound dressings and sponges.

One of the advantages of the immobilized antimicrobial agents is that they are not only antimicrobial at the time of application, but help to minimize contamination by the materials after disposal.

Fibrous materials are also useful in cell culture and tissue engineering devices. Bacterial and fungal contamination is a major problem in eukaryotic cell culture and this provides a safe and effective way to minimize or eliminate contamination of the cultures.

The antimicrobial agents are also readily bound to particles, including nanoparticles, microparticles, millimeter beads, and micelles, that have uses in a variety of applications including cell culture and drug delivery.

B. Implanted and Inserted Materials

The compositions can also be bound to polymeric, metallic, or ceramic substrates. Suitable devices include, but are not limited to surgical, medical or dental instruments, blood oxygenators, ventilators, pumps, drug delivery devices, tubing, wiring, electrodes, contraceptive devices, endoscopes, grafts (including small diameter <6 mm), stents (including coronary, ureteral, renal, biliary, colorectal, esophageal, pulmonary, urethral, and vascular), stent grafts (including abdominal, thoracic, and peripheral vascular), pacemakers, implantable cardioverter-defibrillators, cardiac resynchronization therapy devices, cardiovascular device leads, ventricular assist devices and drivelines, heart valves, vena cava filters, endovascular coils, catheters (including central venous, peripheral central, midline, peripheral, tunneled, dialysis access, urinary, neurological, peritoneal, intra-aortic balloon pump, angioplasty balloon, diagnostic, interventional, drug delivery, etc.), catheter connectors and valves (including needleless connectors), intravenous delivery lines and manifolds, shunts, wound drains (internal or external including ventricular, ventriculoperitoneal, and lumboperitoneal), dialysis membranes, infusion ports, cochlear implants, endotracheal tubes, tracheostomy tubes, ventilator breathing tubes and circuits, guide wires, fluid collection bags, drug delivery bags and tubing, implantable sensors (e.g., intravascular, transdermal, intracranial), wound treatments (sutures, cell scaffolds, bone cements, particles), ophthalmic devices including contact lenses, orthopedic devices (including total and partial hip implants, total and partial knee implants, total and partial shoulder implants, spinal implants (including cervical plates systems, pedicle screw systems, interbody fusion devices, artificial disks, and other motion preservation devices), screws, plates, rivets, rods, intramedullary nails, bone cements, artificial tendons, and other prosthetics or fracture repair devices), dental implants, periodontal implants, breast implants, penile implants, maxillofacial implants, cosmetic implants, valves, appliances, needles, hernia repair meshes, tension-free vaginal tape and vaginal slings, prosthetic neurological devices, tissue regeneration or cell culture devices, or other medical devices used within or in contact with the body or any portion of any of these. The composition can also be in the form of a membrane, nanoparticles, microparticles or beads.

As discussed above, the composition can include additional one or more therapeutic, prophylactic, or diagnostic agents which are released independently of the immobilized antimicrobial. This can be immobilized to or retained in the device, or released from the device, for example, for drug delivery.

The resulting materials are characterized by very favorable properties. For example, where the composition resists >25%, 50%, 75%, 90%, 95%, 99%, 99.9% of the adsorption of protein compared to an untreated control, when placed in contact with biological fluids in vitro. In another embodiment, the composition resists >25%, 50%, 75%, 90%, 95%, 99%, 99.9% of adhered platelets from plasma over a in vitro two-hour flow loop study compared to an untreated control. In still another embodiment, the composition resists >25%, 50%, 75%, 90%, 95%, 99%, 99.9% of thrombus formation by weight over a in vitro two-hour flow loop study compared to an untreated control, as measured by weight over a 14-day intravascular placement in vivo compared to an untreated control, or as measured by thrombus coverage by surface area over a 14-day intravascular placement in vivo compared to an untreated control.

In another embodiment, the composition resists >25%, 50%, 75%, 90%, 95%, 99%, 99.9% of thrombus coverage by surface area over a 30, 60 or 90-day intravascular placement in vivo compared to an untreated control. In another embodiment, the composition resists >25%, 50%, 75%, 90%, 95%, 99%, 99.9% of the adsorption of protein compared to an untreated control, when placed in biological fluid after 30, 60 or 90 days storage in serum

In a preferred embodiment, the composition reduces microbial colonization by >25%, 50%, 75%, 90%, 95%, 99%, 99.9% after 7, 30 or 60 days storage in serum. In the most preferred embodiment, the composition reduces device-associated infection in vivo by >25%, 50%, 75%, 90%, 95%, 99%, 99.9% over the lifetime of the desired device.

### V. Embodiments

In one embodiment, the substrate has immobilized thereon non-fouling polymer brushes with a carbon chain backbone. In a further embodiment the non-fouling polymer brushes possess both non-fouling side moieties and reactive side moieties for subsequent immobilization of membrane targeting antimicrobial agents. In a further embodiment the non-fouling moieties are zwitterions connected to the carbon backbone through stable linkages. In a further embodiment the stable linkages are amides. In a further embodiment, the zwitterions are carboxybetaines, sulfobetaines, phosphorylcholines, or a combination thereof. In a more preferred embodiment, the zwitterions are carboxybetaines. In a further embodiment the reactive side chain moieties are amines (and their derivatives), amides (and their derivatives), carboxylic acids (and their derivatives), azides, maleimides, alkenes, or alkynes. In a further embodiment the reactive side chain moieties are amines. In a further embodiment the membrane targeting antimicrobial agents are immobilized on the polymer brush through direct reaction with the reactive side moieties. In another further embodiment the membrane targeting antimicrobial agents are immobilized on the rective side chain moieties through a linker. In a further embodiment the linker is sulfo-GMBS. In a further embodiment the membrane targeting antimicrobial is Cecropin-A-Melittin.

### EXAMPLES

The present invention will be further understood by reference to the following non-limiting examples.

### Example 1

### Antimicrobial Peptides Immobilized on a Planar Surface Exhibit Antimicrobial Properties

Materials and Methods

A cysteine-incorporating Cecropin-Melittin hybrid peptide (KWKLFKKIGAVLKVLC-NH₂) (SEQ ID NO. 8) was immobilized on a commercial membrane with terminal amine groups (0.340 μmoles of NH₂ per cm², as determined by the picric acid assay) (Intavis Product number 30.100), that is used for the solid state synthesis of peptides. The terminal amine groups of the membrane were reacted with the succinimide groups of sulfo-GMBS and in a subsequent step the maleimide groups of sulfo-GMBS were reacted with the thiol groups of the cysteine-incorporating peptide. This peptide-conjugated membrane was tested for immobilized bactericidal activity against Escherichia coli ATCC 2592.

An overnight culture of a target bacteria in a growth medium such as Cation Adjusted Mueller Hinton Broth, was diluted to approximately 1×10⁵ cfu/ml in pH 7.4 Phosphate Buffered Saline (PBS) using a predetermined calibration between OD₆₀₀ and cell density. A 0.5 cm² sample of immobilized antimicrobial surface was added to 0.75 ml of the bacterial suspension. The sample was covered by the liquid and incubated at 37° C. with a sufficient amount of mixing so that the solid surface is seen to rotate through the liquid. After 1 hour of incubation, serial dilutions of the bacterial suspension were plated on agar plates and allowed to grow overnight for quantifying the viable cell concentration.

Results

Using this procedure, the peptide conjugated membrane produced a 4.2-log reduction of E. coli in solution over 1 h. Testing the amine-functionalized membrane without an antimicrobial peptide conjugated to it for immobilized bactericidal activity did not show a significant reduction in viable bacteria (<0.1 log reduction).

### Example 2

### Antimicrobial Peptides Immobilized on a Planar Surface Exhibit Antimicrobial Properties after More than 3 Weeks Storage in PBS Through Repeated Challenges of Bacteria

Samples identical to those generated in Example 2 were stored at 4° C. in pH 7.4 PBS for more than three weeks. When this peptide-conjugated membrane was tested for immobilized bactericidal activity against Escherichia coli as described in Example 1, an average of a 1.8-log reduction of bacteria in solution occurred over 1 h. The samples were then removed from the testing solution, and placed in fresh PBS. Samples then underwent 10 minutes of ultrasonication, switched to fresh PBS, and underwent an additional 30 minutes of sonication. They were then rinsed and retested. The immobilized antibacterial activity, using the assay described in Example 1, of the washed samples was measured against Escherichia coli ATCC 25922, and an average of a 3.3-log reduction in bacteria occurred in 1 hour. Furthermore, the cidality of the surface was verified by performing Live Dead staining of the cells on the surface, confirming that bacteria contacting the AmP surface were killed upon contact (standard staining procedure BacLight).

### Example 3

### Confirmation that Antimicrobial Activity does not Result from Leached Agent

Materials and Methods

A test was carried out to determine whether the samples used in Example 1 were non-leaching. An evaluation of the supernatant was used to show that the samples used in Example 2 were non-leaching during both rounds of killing before and after washing. 0.4 ml of bacterial solution was removed at the end of the 1 hour incubation between the sample and a solution of bacteria described in Example 2. The 0.4 ml was centrifuged at 3000×g for 5 minutes to remove remaining bacteria. A sample of 0.2 ml of supernatant was removed and added to 0.05 ml of Escherichia coli ATCC 25922 at 5×10⁵ cfu/ml, giving a final concentration of 1×10⁵ cfu/ml, as in the standard antibacterial assay. This mixture was incubated at 37° C. with the same degree of mixing as in the immobilized bactericidal activity assay, and serial dilutions were plated at the end of 1 hour.

Results

The supernatant from both the 1^{st} and 2^{nd} rounds of killing did not show a measurable amount of killing (<0.1-log reduction in viable bacteria). Because the surface demonstrated killing, but the supernatant above the surface does not demonstrate any killing, the immobilized antimicrobial surface is substantially non-leaching.

### Example 4

### The Immobilized Antimicrobial Peptide Surface is Substantially Non-Hemolytic

Materials and Methods

A cysteine-incorporating Cecropin-Melittin hybrid peptide (KWKLFKKIGAVLKVLC-NH2) (SEQ ID NO:8) was immobilized to the amine presenting cellulose membrane with the sulfo-GMBS chemistry as described in Example 2, and the sample was tested to see if it was a substantially non-hemolytic surface. A stock of 10% washed pooled red blood cells (Rockland Immunochemicals Inc, Gilbertsville, Pa.) was diluted to 0.25% with a hemolysis buffer of 150 mM NaCl and 10 mM Tris at pH 7.0. A 0.5 cm² antimicrobial sample was incubated with 0.75 ml of 0.25% red blood cell suspension for 1 hour at 37° C. The solid sample was removed and cells spun down at 6000 g, the supernatant removed, and the OD414 measured on a spectrophotometer.

Total hemolysis is defined by diluting 10% of washed pooled red blood cells to 0.25% in sterile DI water and incubating for 1 hour at 37° C., and 0% hemolysis is defined by a suspension of 0.25% red blood cells in hemolysis buffer without a solid sample.

Results

The peptide immobilized sample produced only 4.95% hemolysis using this assay, demonstrating that the sample is a substantially non-hemolytic surface.

### Example 5

### The Immobilized Antimicrobial Peptide Surface Resists Viable Bacteria Adhesion for 28 Days

Materials and Methods

The ability of the CM modified membrane of example 1 was assessed for its ability to limit adhesion of viable bacteria. Briefly, 10⁷ cfu/ml of S. aureus 10390 were suspended in PBS and incubated for 2 hours with CM modified and unmodified membrane at 37° C. in 750 μl in a 15 ml Falcon® tube. After this time, the sample was rinsed 3× with PBS and sonicated in 750 μl of PBS in a new 15 ml Falcon® tube. The recovered colonies were diluted and plated for enumeration.

Results

The CM modified membranes were stored in PBS for 4 weeks at room temperature and the adhesion tested at various intervals. The adhesion of viable cells was reduced 95% on the CM modified membrane versus controls. The CM modification reduced adhesion 97% at 1 week storage, 97% at 2 weeks storage, 96% at 3 weeks storage, and 94% at 4 weeks storage, demonstrating prolonged efficacy. The storage solution for these samples did not display antimicrobial activity in an assay sensitive to 0.5 ug peptide/ml.

### Example 6

### A D-Amino Acid Version of CM is Equally Active with an L-Amino Acid Version when Immobilized

The adhesion study in example 5 was repeated with D-amino acid CM compared to L-amino acid CM against S. epidermidis 14990. Both samples showed a 99.5% reduction in viable adhered bacteria, indicating the effect is not based on chirality and the peptide is acting through non-specific attack on the membrane.

### Example 7

### The Tethering Chemistries Produce Active Samples with a Variety of Non-Homologous Peptide Sequences

Materials and Methods

Samples were synthesized as in example 1 and tested as in example 5, using peptides CM, MICL1, MICL41, and MICL42. A reduction in viability of 0.7 log or greater was seen for CM, MICL1, MICL41, and MICL42 against S. aureus 10390 and for CM, MICL1, and MICL42 against E. coli 25922. A variety of non-homologous sequences produce broad spectrum activity using various architectures.

### Example 8

### The Immobilized Antimicrobial Peptide Surface is Effective Against an Array of Bacteria, Including Drug Resistant Bacteria

Samples were synthesized as in example 1 and tested as in example 5 against a panel of bacteria. Log reduction in adhesion of 1.8 vs E. coli 25922, 2.3 vs S. aureus 10390, 2.4 vs S. epidermidis 14990, 1.1 vs Methicillin resistant S. aureus 32 (A5984), 1.6 vs Vancomycin resistant enterococcus (A6349), and 2.2 vs Acinetobacter ATCC 49137 (A9934).

### Example 9

### The Immobilized Antimicrobial Peptide Surface is Biocompatible Upon Implantation

Samples were synthesized as in example 1 and implanted in New Zealand white rabbits intramuscularly and subcutaneously. The CM modified samples showed statistically equivalent macroscopic and microscopic biocompatibility scores when compared to a control implant of the unmodified cellulose membrane. All samples were in the biocompatible range.

### Example 10

### The Preparation of Zwitterionic Surfaces

A glass slide was silanized to bind an ATRP initiator on the surface. N-(3-Sulfopropyl)-N-(methacryloxyethyl)-N,N-dimethylammonium betaine (SBMA), or 2-carboxy-N,N-dimethyl-N-(2-methacryloyloxyethyl)ethanaminium inner salt (CBMA) was polymerized on the surface using CuBr as catalyst and 2,2′-bipyridine as ligand. The reaction was performed at room temperature for 4 hours using methanol/water (1:1) as solvent. ELISA was used to test the fibrinogen adsorption on the surfaces. The protein adsorption on surfaces with polySBMA or polyCBMA exhibit more than 95% reduction compared with that on bare glass surfaces.

### Example 11

### Preparation of Amp-Immobilized Carboxybetaine Polymer Brushes

Poly(carboxybeatine acrylamide) (polyCBAA) were grafted on a substrate using surface-initiated atom transfer radical polymerization (ATRP). The substrate (Ti, glass, or silicon) was silanized by a short-chain trialkoxysilane, 2-bromo-2-methyl-N-3-(trimethoxysilyl)propyl-propanamide (BrTMOS). CuBr (1.0 mmol) and the silanized substrate were placed in a 50 ml flask in a dry box under nitrogen protection and sealed with rubber septum stoppers. Degassed solution (pure water and methanol in a 1:1 volume ratio, 10 mL), 2,2′-bipyridine (BPY, 1 mmol), and CBAA (3.8 mmol) were then transferred to the flask using a syringe under nitrogen protection. After reacting for one hour, the substrates were removed and rinsed with ethanol, PBS, and water. The samples were kept in water overnight. The substrate was dried in a stream of nitrogen before use.

The polyCBAA brushes grafted surface was activated by incubating the substrate in a freshly prepared solution containing 2 mg/mL N-hydroxysuccinimide (NHS) and 2 mg/mL 1-ethyl-3-(3-dimethylaminopropyl)-carbodiimide (EDC) in a mixed solvent of dioxane/water (v/v 14:1) for 1 h at room temperature. The antimicrobial peptide (AMP) was linked to the activated surface by putting a 10 μl drop of 2 mg/mL AMP in PBS onto the surface, covering the surface with a glass cover slip, and then incubating the AMP with the activated surface for approximately 24 h at 4° C. in a humid environment. The substrate was then treated with 1 M ethanolamine (pH 8.5) for 10 min to remove any unreacted NHS.

### Example 12

### Preparation of CBAA Block Copolymers

Poly(propylene oxide) (PPO) with a ATRP macroinitiator (PPO-Br) was synthesized by reacting monohydroxybased poly(propylene glycol) with 2-bromoisobutyrylbromide in THF. The product was purified by extraction with brine three times. The carboxybetaine block copolymer was polymerized in 10 mL of methanol using [CBAAA]/[PPO—Br]/[CuBr]/[BPY]) 50:1:1:2 under nitrogen at room temperature. After 24 h, the resulting reaction solution was passed through an aluminum oxide column, precipitated into ethanol, and redissolved into water repeatedly to remove residue catalysts. After solvent evaporation, the copolymer was dried in a vacuum oven at room temperature to yield a white powder.

### Example 13

### Preparation of Crosslinkable Carboxybetaine Coatings

The crosslinkable carboxybetaine copolymer was prepared through a two-step reaction: copolymerization and betainisation. Copolymerization was a normal free radical polymerization with 2-(dimethylamino)ethyl methacrylate (20-80 mol %), n-dodecylmethacrylate (5-50 mol %), 2-hydroxypropyl methacrylate (0-50 mol %) and 3-(trimethoxysilyl)propyl methacrylate (1-5 mol %) using azobisisobutyronitrile (AIBN) as an initiator. The reaction was performed at room temperature for 24 hours under nitrogen protection, then, the copolymer was betainized with β-propiolactone to produce the carboxybetaine copolymer in dried acetone. The precipitate was dissolved in methanol and dialyzed for two days. The polymer solution can be applied on a substrate using a dip-coating method. After treated at 80° C. for two days, the copolymer was crosslinked on the surface, forming a hydrophilic coating.

Unless defined otherwise, all technical and scientific terms used herein have the same meanings as commonly understood by one of skill in the art to which the disclosed invention belongs. Publications cited herein and the materials for which they are cited are specifically incorporated by reference.

We claim:

1. A biocompatible non-fouling antimicrobial medical composition comprising
a substrate,
membrane-targeting antimicrobial agent covalently and stably immobilized to the substrate,
the substrate formed of or including a non-fouling material.

2. The composition of claim 1 wherein the combination of the non-fouling material and covalently immobilized antimicrobial agent increases the degree of non-fouling or antimicrobial activity as compared to substrate having either non-fouling material or covalently immobilized antimicrobial agent.

3. The composition of claim 1 wherein the non-fouling material is a zwitterionic material that forms or is coated onto the substrate, the material comprising a polymer having at least 10%, 30%, 50%, 70%, 90%, or 100% of its units in the form of pendant groups which impart protein adsorption resistance.

4. The composition of claim 1 wherein the membrane-targeting antimicrobial agent is an antimicrobial peptide or peptidomimetic.

5. The composition of claim 4 comprising antimicrobial peptide composed in whole or in part of d-amino acid residues.

6. The composition of claim 3 wherein the antimicrobial agent is covalently bound to the zwitterionic material.

7. The composition of claim 3 wherein the zwitterionic material comprises a pendant group selected from the group consisting of ester, ether, hydroxyl, lactone, lactam, anhydride, carboxylic, unsaturated groups (such as vinyl, allyl, styrene, maleimide, azide, alpha-beta unsaturated carbonyls, maleic anhydride, acryamido, acryloyloxyl, and methacryloyloxyl), amine (including primary, secondary, tertiary and quaternary amine), isocyanate, thiocyante, halide, activated ester, azide, sulfhydryl, chlorosilyl, alkoxysilyl, alkyl, fluoroalkyl, aromatic, ring systems (epoxide, oxirane, cyclopropane, cyclobutane, cyclobutene, aziridine, oxetane, furan, thiphene, pyrrole, pyrrolidine, morpholine, dioxane, thiazole, imidazole diazetidine, dihydroazete, oxathiolane, isoxazole, oxazole, silole, thiadiazine, thiepine, indoles, quinolines, carbazoles), sulfo, phosphoric intermolecular zwitterions, carboxybetaine zwitterions, sulfobetaine zwitterions, phosphorylcholine groups, oligoethers, sugar-based groups, and combinations thereof.

8. The compositions of claim 7 where the pedant group is a hydrogen bond acceptor, but not donor, of less than 500 daltons.

9. The composition of claim 3 wherein the polymer forms a crosslinked matrix or gel.

10. The composition of claim 1 comprising a tether binding the antimicrobial agent to the non-fouling material or to the substrate.

11. The composition of claim 10 wherein the tether is a polymer of molecular weight of greater than 1000, 5000, 7500, or 10000 daltons.

12. The composition of claim 10 wherein the tether is a brush or branched polymer having one end covalently immobilized on the substrate and the branches presenting n sites for attachment of membrane-targeting antimicrobial agents, where n≧1.

13. The composition of claim 1 further comprising a flexible, hydrophilic tether.

14. The composition of claim 1 further comprising a flexible, amphiphilic or hydrophobic tether.

15. The composition of claim 13 wherein the non-fouling material is a tether comprising a polymer having at least 10%, 30%, 50%, 70%, 90%, or 100% of its units in the form of pendant groups which impart protein adsorption resistance.

16. The composition of claim 1 comprising a substrate having immobilized thereon a membrane targeting antimicrobial agent attached through a molecular structure yielding a surface antimicrobial activity (per cm² and/or per mg of active agent) greater than the antimicrobial activity of a uniformly tethered monolayer of the antimicrobial agent.

17. The composition of claim 3 wherein the zwiterionic material is a polymeric brush or branch structure, having one or more sites on the brush or branch for attachment of another molecule.

18. The composition of claim 1 comprising
A first brush or branch non-fouling material not having antimicrobial agent bound thereto, and
A second linear, brush or branch tether or non-fouling material having membrane targeting antimicrobial agent immobilized thereto.

19. The composition of claim 10 wherein the tether utilized to immobilize the membrane targeting antimicrobial is of sufficient length and flexibility to allow interaction of a biologically relevant amount of the antimicrobial with bacteria encountering the surface.

20. The composition of claims 1 wherein the surfaces display antimicrobial efficacy against both gram positive and gram negative bacteria.

21. The composition of claim 1 wherein the composition resists at least 25%, 50%, 75%, 90%, 95%, 99%, or 99.9% of the adsorption of protein compared to an untreated control, when placed in biological fluid for a period of one hour at 37 C.

22. The composition of claim 1 wherein the composition retains at least 25%, 50%, 75%, 90%, 95%, 99%, or 99.9% of the antimicrobial efficacy after 1, 3, 7, 14, or 30 days in vivo.

23. The composition of claim 1 wherein the surfaces are substantially non-hemolytic and non-thrombogenic.

24. The composition of claims 1 wherein the membrane targeting antimicrobial is immobilized at a density >10, 20, 50, 100 mg/cm² or at or below 0.2 mg/cm² of immobilized antimicrobial agent, more preferably at or below 0.1 mg/cm², even more preferably at or below 0.05 mg/cm², and most preferably at or below 0.01 mg/cm²

25. The composition of claim 1 wherein the substrate is constructed from a polymer, ceramic, metal, or combination thereof.

26. The composition of claim 1 wherein the substrate is an implantable or insertable medical device.

27. The composition of claim 1 wherein the substrate is a fibrous material for wound dressing, surgical drapes or clothes, tissue engineering, tampanade, or feminine hygiene.

28. The composition of claim 26 wherein the substrate is a catheter, orthopedic device, cardiac rhythm management device, cardiac rhythm management lead, or stent.

29. The composition of claim 1 wherein one or more bioactive agents are immobilized on or contained within the substrate in addition to the membrane targeting antimicrobial agent.

30. The composition of claim 29 wherein the additional bioactive agent promotes cell adhesion, cell growth, tissue endothelialization, or is anti-thrombotic.

31. The composition of claim 30 wherein the additional bioactive agent is an adhesion peptide RGD or heparin or fragment thereof.

32. The composition of claim 29 wherein the additional bioactive agent is patterned on the substrate.

33. The composition of claim 1 wherein the antimicrobial agent is resistant to protein adsorption.

34. The composition of claim 33 wherein the amino acid residues imparting the protein adsorption resistance are non-natural residues containing intramolecular zwitterions.

35. A method for making the composition of any of claims 1-34 comprising modifying the surface of the substrate through a gas-phase technique selected from the group consisting of plasma, corona discharge, flame treatment, UV/ozone, UV only, ozone only, electrolytic treatment, oxidation, silanization, anodizing, aminolysis, hydrolysis, reduction, activation of alcohol chain ends with tosyl chloride and subsequent chemistry, graft copolymerisation of vinyl compounds by chemical initiation, or ion beam treatment in the presence of vinyl monomers.

36. The method of claim 35 wherein the surface of the substrate is modified to present reactive groups for coupling of antimicrobial agents, or tethering structures for antimicrobial agents, through coextrusion of a polymeric material containing reactive groups for the attachment.

37. The method of claim 36 wherein the surface of the substrate is modified to present reactive groups for coupling of antimicrobial agents, or tethering structures for antimicrobial agents, through self segregation of hydrophilic or hydrophobic end groups covalently attached to the base polymeric material.

38. The method of claim 36 wherein the surface of the substrate is modified to present reactive groups for coupling of antimicrobial agents, or tethering structures for antimicrobial agents, through dip coating of the substrate with a solution of a polymer possessing a multiplicity of reactive groups for the attachment.

39. The method of claim 36 wherein the substrate surface is treated to introduce groups on the substrate surface, which can react with functional groups on the peptide, wherein the groups on the substrate are selected from the group consisting of ester, ether, hydroxyl, epoxy, lactone, lactam, anhydride, carboxylic, unsaturated groups (such as vinyl, allyl, styrene, maleimide, azide, alpha-beta unsaturated carbonyls, maleic anhydride, acryamido, acryloyloxyl, and methacryloyloxyl), amine (including primary, secondary, tertiary and quaternary amine), isocyanate, thiocyante, halide, activated ester, azide, sulfhydryl, chlorosilyl, alkoxysilyl, alkyl, fluoroalkyl, aromatic, ring systems (epoxide, oxirane, cyclopropane, cyclobutane, cyclobutene, aziridine, oxetane, furan, thiphene, pyrrole, pyrrolidine, morpholine, dioxane, thiazole, imidazole diazetidine, dihydroazete, oxathiolane, isoxazole, oxazole, silole, thiadiazine, thiepine, indoles, quinolines, carbazoles) sulfo, phosphoric, intermolecular zwitterions, carboxybetaine zwitterions, sulfobetaine zwitterions, phosphorylcholine groups, oligoethers, sugar-based groups, and combinations thereof.

40. The method of claim 35 wherein thiol or amino groups in the antimicrobial agent can react directly by conjugate addition reaction with unsaturated groups selected from the group consisting of maleimides, vinyl sulfones, acrylamides and acrylates present in or on the substrate.

41. The method of claim 35 wherein the antimicrobial agent is bound to the substrate by a functional group present in the peptide selected from the group consisting of amine, thiol, carbonyl, carboxyl, aldehyde, vinyl, phenyl, phenol and hydroxyl.

42. The method of claim 35 wherein one or more amine, alcohol or thiol groups on the antimicrobial agent is reacted directly with a functional group on the surface of the substrate selected from the group consisting of carboxyl, hydroxyl, isocyanate, isothiocyanate, acyl azide, N-hydroxysuccinimide ester, aldehyde, epoxide, anhydride, halides, sulphydryl, vinyl, and lactone.

43. The method of claim 35 where one or more free amino, anhydride, carboxylic, sulfhydryl or hydroxyl groups of the antimicrobial agent are attached to a surface containing epoxide functional groups.

44. A method of making the composition of claim 1 comprising dipcoating a substrate with a non-fouling material comprising the membrane targeting antimicrobial agent.

45. An immobilized antimicrobial peptide comprising one or more non-natural amino acids.

46. The antimicrobial peptide of claim 44 comprising one or more D-amino acids.
